(19) United States
(12) Patent Application Publication
(10) Pub. No.: US 2003/0054340 A1
(43) Pub. Date: Mar. 20, 2003

(54) Neural tracing with a conditional herpesvirus and methods and uses thereof

(76) Inventors: Jeffrey M. Friedman, New York, NY (US); Jeff DeFalco, Ardsley, NY (US); Lynn W. Enquist, Princeton, NJ (US); Mark Tomishima, Princeton, NJ (US)

Correspondence Address: KLAUBER & JACKSON, 411 HACKENSACK AVENUE, HACKENSACK, NJ, 07601

(21) Appl. No.: 10/109,534

(22) Filed: Mar. 28, 2002

Related U.S. Application Data
(60) Provisional application No. 60/279,954, filed on Mar. 29, 2001.

(51) Int. Cl. C12Q 1/70; C12Q 1/00

(52) U.S. Cl. 435/5; 435/4

(57) ABSTRACT

The present invention relates to methods for tracing or characterization of neural circuits or neural connections across synapses. The invention relates to recombinant neurotropic viruses which are useful in tracing or characterizing neural connections across multiple synapses.

### RELATED APPLICATIONS

The present application claims the benefit of the filing date pursuant to 35 U.S.C. §119(e) of copending application Serial No. 60/279,954 filed Mar. 29, 2001, which is incorporated herein by reference in its entirety.

### GOVERNMENTAL SUPPORT

The research leading to the present invention was supported, at least in part, by a grant from U.S. Government Granting Agency, Grant No. R01 DK41096 and R01 133506. Accordingly, the Government may have certain rights in the invention.

### FIELD OF THE INVENTION

The present invention relates generally to tracing of neural circuits or neural connections across synapses. The invention also relates to recombinant neurotropic viruses which are capable of tracing of neural connections across multiple synapses.

### BACKGROUND OF THE INVENTION

Pseudorabies virus (PRV) is a member of the neurotropic Alphaherpesvirus subfamily which also includes the human pathogens herpes simplex virus types 1 and 2 (HSV-1 and -2) and varicella-zostervirus (VZV) and the animal pathogens bovine herpesvirus type I (BHV-1), equine herpesvirus type I (EHV-1), andfeline herpesvirus type I (FHV-1). PRV establishes a latent infection in its natural host, the adult swine, but causes a lethal encephalitis in young piglets and in a wide variety of mammals and birds (Ben-Porat, T. and Kaplan, A. S. (1985) in B. Roizman (ed) “The Herpesviruses” (Plenum Publishing Corp., N.Y.) pp. 105-173; Mettenleiter, T. C. (1991) Comp. Immunol. Microbiol. Infect. Dis. 14:151-163). PRV displays a striking neurotropism for both the central and peripheral nervous systems, which it enters by primary infection of cells lining mucosal or epithelial surfaces (Field, H. J. and Hill, T. J. (1974) J. Gen. Virol 23:145-157; Field, H. J. and Hill, T. J. (1975) J. Gen. Virol 26:145-148). Once PRV has invaded the nervous system, it is capable of spreading between chains of synaptically connected neurons in pathways consistent with known neuronal connections (Goodpasture, E. W. and Teague, O. (1923) J. Med. Res. 44:139-184; Kristensson, K. (1996) Brain Res. Bull. 41:327-333; Sabin, A. B. (1938) Proc. Soc. Exp. Biol. Med. 38:270-275). The spread of PRV between neurons is confined to sites of synaptic contact and can occur in both the anterograde and retrograde directions (reviewed in Card, J. P. (1998) Anat. Rec. (New Anat.) 253:176-185 and Enquist, L. W. et al. (1999) Adv. Vir. Res. 51:237-247).

Neurotropic alphaherpesviruses can replicate within postmitotic neurons and produce infectious progeny that pass transneuronally to infect other synaptically linked neurons (Enquist, L. W. et al. (1999) Adv. Virus Res. 51:237-347; Kristensson, K. (1996) Brain Res. Bull. 41:327-333). This self-amplifying spread in neurons has been exploited by a number of investigators to gain further insight into the organization of neuronal circuitry in the mammalian brain and neurotropic alphaherpesviruses have become popular tools for transynaptic analysis of neural circuitry (Card, J. P. (1998) Neurosci. Behav. Rev. 22:685-694; Enquist, L. W. and Card, J. P. (1996) in Lowenstein and Enquist, ed. “Protocols for Gene Transfer in Neuroscience” (John Wiley and Sons, UK), pp. 333-348; Loewy, A. D. (1995) in M. G. Kaplitt and A. D. Loewy (ed.) “Viral Vectors: Gene Therapy and Neuoscience Applications” (Academi Press) pp. 349-366: Ugolini, G. (1995) ibid pp. 293-318; Strick, P. L. and Card, J. P. (1992) in J. P. Bolar (ed.) “Experimental Neuroanatomy: A Practical Approach” pp. 81-101).

The use of neurotropic alphaherpesviruses has greatly advanced our ability to visualize ensembles of neurons that contribute to multisynaptic circuits in the central nervous system (CNS) (Card, J. P. (1998) Anat. Rec. 253:176-185). In particular, the attenuated vaccine strain of pseudorabies virus (PRV-Bartha) has been used successfully as a self-amplifying neural tracer after peripheral application or direct injection into brain parenchyma (Card, J. P. et al. (1999) J. Comp Neurol 407:438-452; Chen, S. et al (1999) Brain Res. 838:171-183; Enquist, L. W. et al. (1999) Adv. Virus Res. 51:237-247). The usefulness of PRV as a neuraltracer relies on its ability to infect chains of hierarchically connected neurons via specific transsynaptic passage of progeny virus rather than infection by lytic release into the extracellular space (Enquist, L. W. et al. (1999) Adv. Virus Res. 51:237-347; Kuypers, H. G. et al. (1990) Trends Neurosci 13:71-75). Typically, PRV infects the CNS by invading neurons in the periphery and then replicating and spreading to the CNS via synaptically linked neurons. However, PRV can also invade neurons through their somata if the viral concentration is sufficient (Card, J. P. (1998) Neurosci. Biobehav. Rev. 22:685-694), as evidenced by primary infection of retinal ganglion cells (RGCs) after intravitreal injection of PRV (Card, J. P. et al. (1991) Neuron 6:957-969; Moore, R. Y. et al. (1995) J. Comp. Neurol. 352:351-366; Provencio, I. et al. (1998) J. Comp. Neurol. 395:417-439). Infection of RGCs with PRV-Bartha, followed by viral replication, results in the anterograde transsynaptic infection of a restricted set of retinorecipient neurons [i.e., suprachiasmatic nucleus (SCN), intergeniculate leaflet (IGL), pretectum (PT), and lateral terminal nucleus]. Intravitreal injection of the wild-type virus, PRV-Becker, produces transneuronal infection of neurons in all retinorecipient subcortical regions (Card, J. P. et al. (1991) Neuron 6:957-969). The factors that determine the specificity of PRV-Bartha infection of selective retinorecipient targets are not completely understood, although deletion of specific genes in PRV-Becker results in a restricted neurotropism identical to that demonstrated with PRV-Bartha (Whealy, M. E. (1993) J. Virol. 67:3786-3797).

Recombinant viruses that express unique gene products as reporters of infection are useful tools for defining connections among neurons (Jansen, A. S. P. et al. (1995) Science 270:253-260; Levatte, M. A. et al. (1998) Neuroscience 82:1253-1267). In a notable application of this experimental approach, Jansen and colleagues injected two genetically modified forms of the Bartha strain of pseudorabies virus (PRV-Bartha) into peripheral targets innervated by separate populations of spinal cord neurons (Jansen, A. S. P. et al. (1995) Science 270:253-260). Transynaptic infection of CNS neurons by both strains of virus was demonstrated, but the percentage of animals that exhibited dual-infected neurons was remarkably small. For example, only 20 of 256 animals exhibited productive replication of both viruses, and of those, only 8 were viewed as containing a specific pattern of infection worthy of analysis. It is likely that the low infection rate in this experiment is due, at least in part, to the use of titers of virus that were considerably below the 50% lethal dose (LD₅₀) for PRV-Bartha in rats. However, other factors may have contributed to the low frequency of neuronal coinfection.

Although viral transsynaptic tracing represents an important methodological advance for the analysis of CNS circuits, functional analysis of virus-infected neurons has been limited to sensory or sympathetic ganglia in culture (Kiraly, M. and Dolivo, M. (1982) Brain Res. 240:43-54; Mayer, M. L. et al. (1986) J. Neurosci 6:391-402) because of the inability to identify virus-infected neurons in situ. Analyses of electrophysiological properties of neurons, in the context of known functional connections of the recorded neuron, would represent a further important methodological advance for the analysis of CNS circuits.

The development of retrogradely transported fluorescent tracers has allowed investigators to examine the physiology of neurons with known projections (Katz, L. C. et al. (1984) Nature (London) 310:498-500; Ramoa, A. S. et al. (1987) Science 237:522-525; Viana, F. (1990) Neuroscience 38:829-841). However, such studies usually require direct and accurate injection of a target region followed by retrograde transport to identify first-order neurons projecting to the target. Other “prelabeling” studies have used constructs of green fluorescent protein to label neurons that possess a particular genetic phenotype, such as expression of gonadotropin-releasing hormone (Spergel, D. J. et al. (1999) J. Neurosci. 19:2037-2050). Both of these techniques have allowed examination of the physiological properties of neurons in vitro that possess presumed anatomical or functional correlates in the intact animal.

One of the challenges facing neurobiologists is mapping the connections between neurons within the mammalian brain. Reagents currently available only allow determination of connections between gross brain regions, yielding little information regarding the connections made to specific neuron types. Therefore, in view of these deficiencies attendant with prior art methods, it should be apparent that there exists a need in the art for a method or technology allowing one to trace neural circuits or specifically infect cells across neural circuits involving neurons that express particular genes or that make a particular neurotransmitter, neuropeptide or receptor.

The citation of references herein shall not be construed as an admission that such is prior art to the present invention.

### SUMMARY OF THE INVENTION

The invention provides a method for characterizing neural circuits and neural connections. The invention provides a method for tracing of neural circuits or neural connections across synapses.

The present invention provides a method for characterizing a neuronal pathway in an animal which utilizes a neurotropic virus capable of spreading between synaptically connected neurons which is conditional for marker peptide expression, whereby said marker peptide is expressed upon a specific recombination event and/or in the presence of an inductive agent. In the method, the neurotropic virus is introduced, followed by introduction or activation of a recombinase or inductive agent, such that the marker peptide is expressed and can be monitored. In as much as the neurotropic virus spreads between synaptically connected neurons and its presence and/or replication is indicated by the marker peptide, the neural circuit or neural connections across synapses can be characterized by assessing or monitoring marker peptide expression.

The invention provides a method for characterizing a neuronal pathway in an animal comprising:

a) introducing a neurotropic alphaherpesvirus capable of spreading between synaptically connected neurons which is conditional for marker peptide expression, whereby said marker peptide is expressed upon a specific recombination event;

b) introducing or activating a recombinase such that said recombination event is induced and marker peptide expression occurs; and

c) monitoring marker peptide expression.

The method of the present invention contemplates utilizing a neurotropic alphaherpesvirus which is also conditional for replication, whereby replication is permissive upon a specific recombination event, further comprising:

a) introducing or activating a recombinase such that said recombination event is induced and replication of said neurotropic virus occurs.

In one contemplated method of the present invention, both marker protein expression and replication are conditional upon the same specific recombination event.

The invention further provides a method for characterizing a neuronal pathway in an animal comprising:

a) introducing a neurotropic alphaherpesvirus capable of spreading between synaptically connected neurons which is conditional for marker peptide expression, whereby said marker peptide is expressed in the presence of an inductive agent;

b) introducing said inductive agent such that marker peptide expression occurs; and

b) monitoring marker peptide expression.

In a variation on this method, the neurotropic alphaherpesvirus is also conditional for replication, whereby replication is permissive upon a specific recombination event, and the method includes introducing a recombinase such that said recombination event is induced and replication of said neurotropic virus occurs.

The method of the present invention may utilize a recombinase from prokaryotic or eukaryotic cells. The invention particularly provides for the use of a recombinase selected from the group of Cre, FLP, X integrase, R recombinase or Kw recombinase.

The method of the invention includes methods wherein the expression of the recombinase is under the control of a heterologous expression control sequence. The heterologous expression control sequence may be expressed in particular cell types, may respond to particular inductive agents, or may be constitutively expressed.

In a particular method of the present invention the heterologous expression control sequence controlling the recombinase is neural cell specific. In a further particular method of the present invention, the heterologous expression control sequence is that of a neuropeptide or neuroactive peptide. The heterologous expression control sequence for use in the method of the invention may be selected from the group of NPY and Ob-R.

In the method of the present invention, the neurotropic alphaherpesvirus may be introduced by intranasal infection. In a further method of the invention, the neurotropic alphaherpesvirus may be introduced by injection into the brain of said animal. In addition, or alternatively, the neurotropic alphaherpesvirus may introduced by injection or infusion into the spinal cord of said animal. Still further contemplated are methods wherein the neurotropic alphaherpesvirus is introduced by injection into a peripheral organ of said animal.

The invention also relates to recombinant neurotropic viruses which are capable of tracing of neural connections across multiple synapses. The neurotropic viruses of the present invention, particularly neurotropic alphaherpesviruses, are capable of conditionally expressing a marker peptide. The neurotropic virus conditionally expresses the marker peptide upon a specific recombination event and/or in the presence of an inductive agent.

The invention provides a recombinant neurotropic alphaherpesvirus capable of conditionally expressing a marker peptide, wherein said marker peptide is expressed upon a specific recombination event and/or in the presence of an inductive agent.

The neurotropic virus of the present invention includes a pseudorabies virus.

The neurotropic alphaherpesvirus of the present invention can be derived from any strain of PRV, and may particularly derived from a PRV strain selected from teh group of Bartha, Becker or Kaplan. The present invention particularly provides a neurotropic alphaherpesvirus which is selected from the group of Ba2000 or Ba2001.

The invention further provides a transgenic animal harboring a neurotropic alphaherpesvirus capable of spreading between synaptically connected neurons which is conditional for marker peptide expression.

The invention also includes a transgenic animal harboring a neurotropic virus which is conditional for marker peptide expression and which is also conditional for replication.

Other objects and advantages will become apparent to those skilled in the art from a review of the following description which proceeds with reference to the following illustrative drawings.

### BRIEF DESCRIPTION OF THE DRAWINGS

FIG. 1 Construction of recombinant PRV strain Ba2001. (A) Diagram of Ba2001. The parent virus, PRV-Bartha Blue (BaBlu) carries a deletion in the tk gene. The elements of the targeting construct pG2LTHTK are shown below the site of the region of recombination into the gG region of Bartha Blue. In the targeting construct, the CMV promoter drives the synthesis of tau-GFP and TK. In this bicistronic message TK is translated from an IRES element. In this configuration, RNA synthesis is interrupted by a LOX stop cassette that includes a polyadenylation site. In the absence of cre the message is cleaved and no translation occurs. After cre mediated excision of the Stop cassette, expression of GFP and TK is activated. (B) Southern blot analysis of Ba2001. Nucleocapsid DNA was digested with endonuclease(s) listed above each lane and the blots hybridized with the probe listed below each autoradiograph. The sizes of expected restriction fragments using GFP or tk as probes are shown and match the sizes of the fragment in Panel (A). (C) Strategy for tracing afferent connections of ObRb expressing neurons. The experiment is performed using mice that express Cre only in ObRb expressing neurons. ObRb is the signaling form of the leptin receptor. In an ObRb-Cre mouse, Ba2001 would infect two parallel circuits, one containing an ObRb expressing neuron that co-expresses Cre recombinase while the other circuit does not. After infection of the ObRb/Cre neuron only, Ba2001 is modified by Cre, resulting in activation of both Tau-GFP and TK expression. This in turn allows DNA replication and subsequent retrograde infection of afferent neurons. The MC4 Receptor neurons do not express Cre and are non-permissive for both viral replication (Tenser, R. B. et al (1983) J Gen Virol 64:1369-1373) and marker protein expression. This strategy allows the specific visualization of the ObRb circuit.

FIG. 2 Cre-dependent expression of Tau-GFP by Ba2001. (A) Conditional expression of tau-GFP in tissue culture cells. Ba2001 was passed through either PK15 cells (left panel) or TE-Cre, a Cre-expressing cell line (right panel). GFP expression is observed only after excision of the LOX stop cassette, right panel. (B) Cre-dependent GFP expression in SynI-Cre mice. Ba2001 was injected intracranially into wild type C57B1/6 (left panels) or C57B1/6 Syni-Cre Tg mice (right panels). Tau-GFP was detected by anti-GFP immunofluorescence (top panels) and the expression of viral late protein gC was detected using anti-gC antibodies(bottom panels). Magenta particles are fluorescent microspheres that were co-injected with the virus to mark the point of injection. Viral replication and expression of GFP were observed in the Syn1-cre but not in the wild type mice. (C) Ba2001 ocular injection. 10⁵ pfu of Ba2001 was injected unilaterally into the vitreous humor of CMV-Cre transgenic mice. Animals were sacrificed at 5 days post-infection, brains sectioned and GFP expression visualized with anti-GFP immunofluorescence. Tau-GFP expression was observed in the Periaqueductal Grey (PAG), Edinger Westphal Nucleus (EW), Amygdala (BMA) and Paraventricular nucleus (PVN) all of which are known to project through one or more synapses to the eye.

FIG. 3 Specificity of Cre Recombination in NPY expressing cells in vivo. The in vivo requirement of Cre for excision of the LOX-STOP cassette was tested in mice that express Cre only in NPY expressing neurons. A TK-virus that was conditional only for GFP was used to infect a transgenic mouse that carried a 150 kB BAC in which cre had replaced the NPY coding region. (A) Replication defective virus Ba2000. This virus is identical to Ba2001 except that the expression cassette does not include the TK gene. As a consequence this virus cannot replicate in non-mitotic cells but still requires Cre for expression of GFP. Following infection of Cre-expressing neurons and deletion of the STOP cassette, Ba2000 expresses TauGFP but remains replication-defective and is unable to spread to higher order neurons. This strategy allows first order neurons to be visualized exclusively. (B) A BAC transgenic mouse was developed in which the NPY gene was replaced by cre. A nuclear localization sequence—Cre open reading frame (NLS-Cre)—polyadenylation sequence (polyA) was inserted 5′ to the ATG in the first exon of the NPY gene. This BAC includes 30 kB of 5′ upstream and 120 kB of downstream sequence and leads to expression of cre in NPY neurons. (C) GFP expression is limited to NPY-expressing neurons. Ba20OO was injected into the somatosensory 2 cortex of NPY-cre transgenic mice. Sections were stained for NPY or GFP. An overlay of the NPY- and GFP-immunoflourescence images (left panel) indicates that GFP expression is only observed in neurons that express NPY (yellow cells). Scale bars=100μ.

FIG. 4 Ba2001 infection of NPY-Cre mice following injection into the arcuate nucleus. NPY-Cre transgenic mice were sacrificed at 4 days after injection of 1.1×10⁶ pfu of Ba2001 into the arcuate nucleus. (A) Viral spread within the posterior hypothalamus. GFP was expressed in the following hypothalamic structures as indicated: ARC, arcuate nucleus; DMH, dorsomedial nucleus; LH, lateral hypothalamus; VMH, ventromedial hypothalamus; SOX, supraoptic decussation; 3V, third ventricle. Scale bar=1001μ. (B) Viral spread to the medial dorsal thalamus (MD). Arrows indicate locations of GFP fluorescent neurons. Scale bar=25μ. (C) Viral spread to the dentate gyrus (DG). Scale bar=25μ. (D) GFP expression in the Pir and ventral basolateral amygdala (BLV) at 5 days post-infection. Scale bar=25μ.

FIG. 5 Ba2001 infection of ObRb-IRES-Cre recombinant mice. A total of 1.3×10⁶ pfu of Ba2001 was injected into the arcuate nucleus of ES-cell derived mice that express Cre from an IRES element inserted in to the 3′ untranslated region of the ObRb gene. Animals were sacrificed post-infection at the times indicated. Brains were sectioned and GFP expression was visualized by anti-GFP immunofluorescence (see panels B-E). Low power (10×) and high power (40×) images are shown on the left and right panels respectively. (A) ObRb IRES-Cre targeting construct. A cassette containing an IRES-NLS-Cre and neo gene, flanked by frt sites, was inserted immediately 3′ to the stop codon in the last exon of ObRb. This cassette was then used for recombination in ES cells and positive recombinant ES clones used to generate the ObRb “knock-in” mouse. (B) Expression of GFP in the hypothalamus at 3 days post-infection. At 3 days, GFP was expressed in the arcuate, DMH, LH and VMH. Higher magnification of the DMH (right panel) revealed a possible connection between two DMH neurons (white arrow). Scale bars: left, 2001; right, 25μ. (C) GFP expression in the limbic and cortical brain regions. GFP expression in the BLA, Pir and LEnt at 5 days post-infection (left). Population of GFP-expressing neurons in the basolateral amygdala (BLA) (right). Scale bars: left, 200μ; right, 25μ. (D) GFP expression in the retrosplenial cortex at 7 days post-infection. GFP expressing neurons in the granular retrosplenial and agranular cortex. Right panel illustrates cells with the morphology of pyramidal cells (red arrows) and interneurons (white arrow). Scale bars: left, 200μ; right, 50μ.

FIG. 6 depicts the overall scheme. The Ba2001 construct is shown. In the presence of cre, recombination at LoxP allows transcription from the CMV promoter, driving tau-GFP and TK.

FIG. 7 depicts alternative bicistronic constructs. (A) In this construct, the first cistron consists of the thymidine kinase open reading frame (ORF). Immediately 3′ to the TK ORF is an IRES (Internal Ribosome Entry Site) which allows for CAP-independent ribosome translation initiation followed by sequence that codes for a Tau-GFP fusion protein. (B) Similar to (A) except that the first cistron codes for Tau-GFP and the second cistron codes for TK.

FIG. 8 depicts alternative monocistronic constructs. (A) Consists of two conditional transcription units both of which utilize a lox-STOP-lox cassette and are thus dependent on Cre recombinase. The first cassette utilizes a CMV promoter which will drive expression of the tau-GFP marker protein. The second cassette utilizes a RSV promoter to drive expression of thymidine kinase. The two loxP sites in the first cassette (CMV-loxP-STOP-loxP-TauGFP-BGH polyA) have been mutated to prevent recombination with the loxP elements contained in the second cassette. These mutations were made in the spacer region of each of the two loxP elements and do not interfere with Cre mediated recombination between two identical elements that contain these alterations. Thus, Cre mediates removal of the STOP cassettes, allowing TauGFP and TK expression. (B) Here, both complementation of the thymidine kinase deficiency and visualization is accomplished by a single TauGFP-TK fusion protein. This protein consists of three functional “domains”. The Tau domain allows for transport of the protein to the dendrites and termini; the GFP domain permits visualization; the TK domain rescues replication deficiency. (C) Thymidine kinase deficiency is complemented by a Tau-TK-HAg fusion protein which also serves as a marker protein through the hemagglutinin epitope tag (HAg). Expression of the fusion protein in neurons can be detected using Immunohistochemistry (IHC) or immunofluorescence (IF). (D) and (E) These two strains are conditional for marker gene expression only and are both on a TK+ background. For (D), a Tau-HAg fusion protein allows identification of neurons infected by a Cre-modified virus via IHC or IF. In (E), neurons become labeled by conditional expression of Tau-GFP. Both of these viral strains can productively infect neurons that do not express Cre recombinase.

FIG. 9 depicts an alternative scheme utilizing a tet and cre responsive system. The background virus contains deletions in both IE180 genes which renders the virus completely replication defective. The inserted cassette contains three transcription units. The first expresses TET-OFF under the control of the RSV promoter. In the absence of tetracycline, TET-OFF activates transcription from promoters that contain the tet operator sequence. The second transcription unit is CMV-Lox-STOP-Lox-TauGFP IRES-TetOFF. The second transcription unit is composed of a tet-responsive promoter driving expression of the IE180 gene. Thus, in the presence of Cre recombinase, the Stop cassette in the first transciption unit is removed, allowing expression of both the TauGFP marker protein and also the TetOFF transcription factor. In the absence of tetracycline, TetOFF activates expression of IE180, which activates viral gene expression and ultimately replication. Through the introduction of tetracycline, or one of the appropriate derivatives, the expression of IE180 can be controlled, in effect allowing the infection to be halted at a particular stage. This would be particularly useful for the purpose of studying infected afferent neurons.

FIG. 10 depicts an alternative conditional scheme. Here the transgenic mouse expresses Cre in a neuron specific manner (ObRb is shown as an example). In addition, the mouse contains two transgene insertions. The first is composed of a CMV-STOP-GFP which allows cre-conditional expression of blue GFP and thus visualization of cre expressing neurons. The second transgene consists of a bidirectional tet-responsive promoter driving expression of both Tet-ON and IE180. Stop cassettes flank either side of the promoter, giving cre-dependent expression of Tet-ON and IE180. Following removal of stop signals by cre, Tet-ON is expressed at basal levels. In the presence of tetracycline, Tet-ON positively autoregulates its own expression as well as IE180 expression. Control over Tet-ON and IE180 expression is desirable because of possible cytotoxic effects from their activation domains. The virus contains deletions in both IE180 genes, rendering it unable to replicate. This virus also contains a CMV-Stop-Tau-TopazGFP cassette inserted into the gG region. After infecting a neurons that expresses cre recombinase in the recombinant/transgenic animal described above, the viral Stop signals are removed allowing expression of the Topaz GFP fusion protein. Since a Cre-expressing neuron will also be expressing IE180 (in the presence of tetracycline), the virus's deficiency will be complemented, allowing viral gene expression and replication to proceed. This in turn leads to the production of infectious viral particles which can then infect second order afferent neurons. Provided that second order neurons do not express cre/IE180, the virus will not be able to replicate in second order neurons. The virus will, however, be able to express Topaz since the Stop signals were permanently removed. The net result would be as follows: Infected Cre-expressing neurons will express both Blue and Topaz GFP, making them readily identifiable; second order neurons will express only Topaz GFP and would be easily distinguished from first order neurons.

FIG. 11 depicts a conditional lethal virus construct. Like the strains that conditionally express either TK or IE180 and GFP, this construct was designed to specifically infect neurons that co-express cre recombinase and a particular gene of interest and retrogradely infect the afferent portion of the circuit. However, in addition to GFP and an essential viral gene (TK/IE180), this strain would also express an attenuated form of diptheria toxin in a cre-dependent manner. Furthermore, the use of a Tet-responsive promoter in tandem with the Tet-ON protein (expressed by the first cistron of this transcription unit) permits control over the expression of diptheria toxin. Following infection of a cre-expressing neuron in the absence of tetracycline/doxycyline, the infection would proceed just as it would for the above conditional TK/GFP viruses. Transcription of the Tet-ON/aDTA open reading frames would occur at basal levels which should not have an effect on cellular metabolism (as shown in similar studies). The positioning of the aDTA ORF in the second cistron would help to minimize its expression. Following administration of TET, the Tet-ON transactivator would become allosterically activated, resulting in the activation of diptheria toxin expression. This in turn would halt all protein synthesis in the cell resulting in the cell′ s death. Whether any infectious viral particles would be produced by this infection would depend on the timing of the tetracycline adminstration (and hence the activation of aDTA expression) relative to the life cycle of the virus. If infectious particles were produced, any subsequent infections would result in the rapid death of the newly infected cell very early in the virus life cycle.

The net effect of this approach would be that infections from virus that have not been modified by cre (that haven't infected a Cre-expressing neuron) wouldn't express either GFP, TK/IE180, Tet-ON or aDTA. These infections would still be “dead-ends” for the virus and would not have deleterious effects on the neurons. Cells that were still alive following infection by a Cre-modified virus would, following Tet administration, be killed outright by the expression of aDTA. This would effectively terminate the infection in the animal so long as Tet was present. The result would be the destruction of a portion of the neural circuit that was afferent to the cre-expressing neurons. The extent of the destruction would depend on the timing of the tetracycline administration relative to the injection of the virus into the animal. Analysis of the resulting phenotype of such an animal could lead to insight regarding the function of the affected neurons.

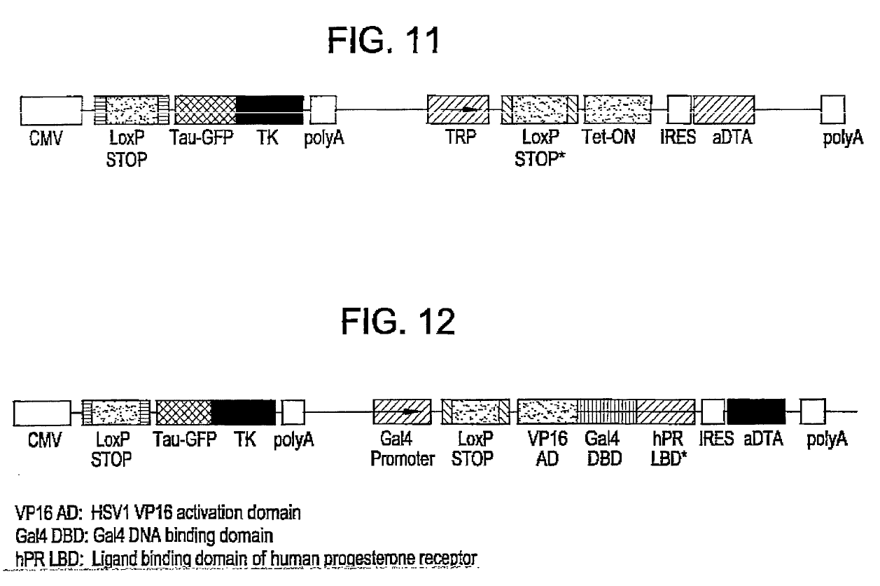

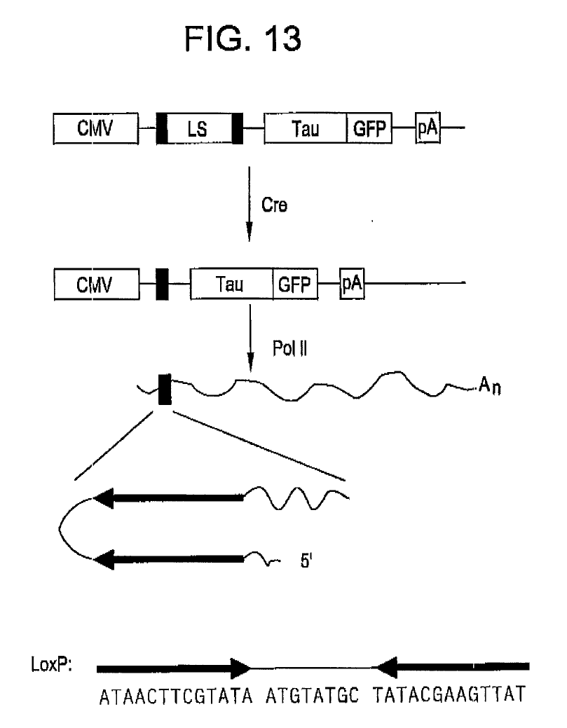

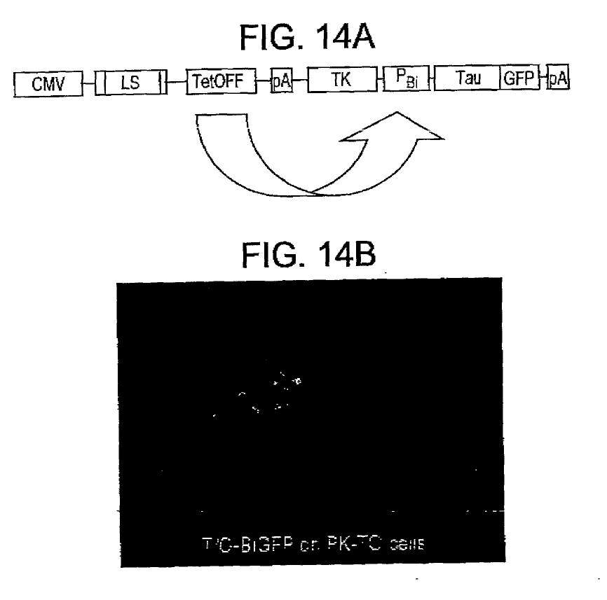

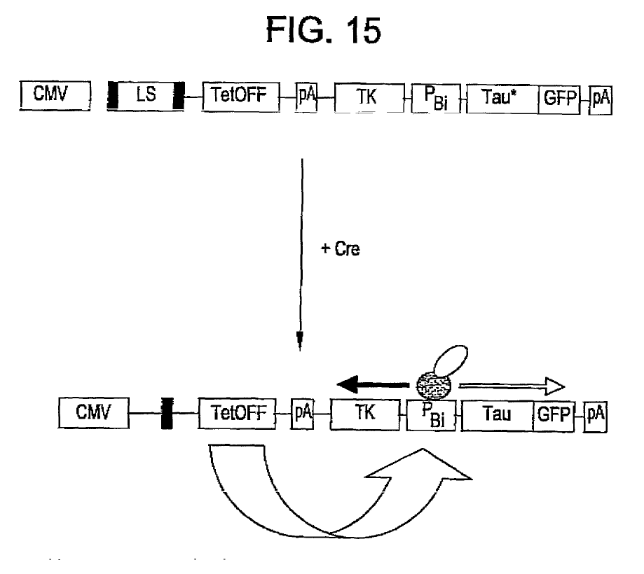

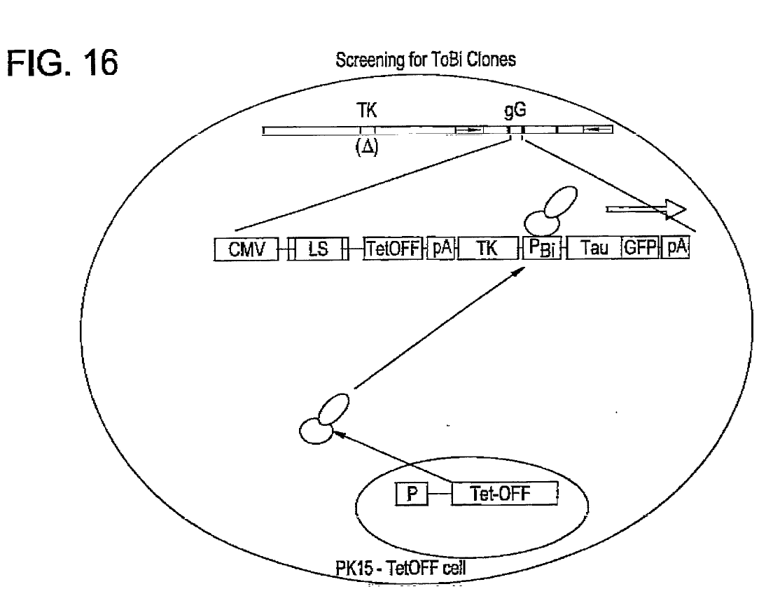

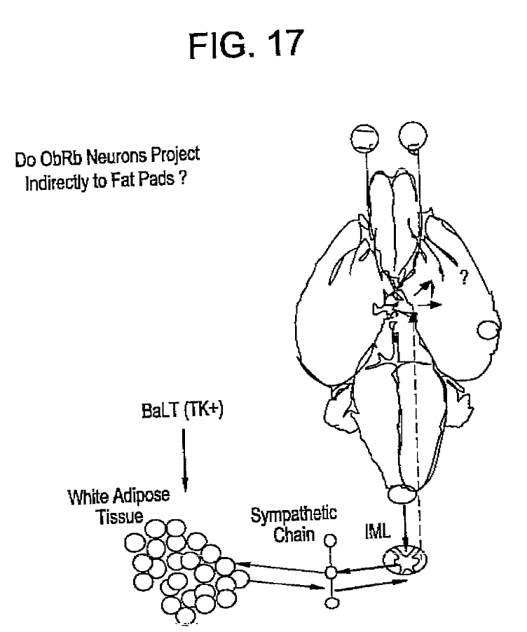

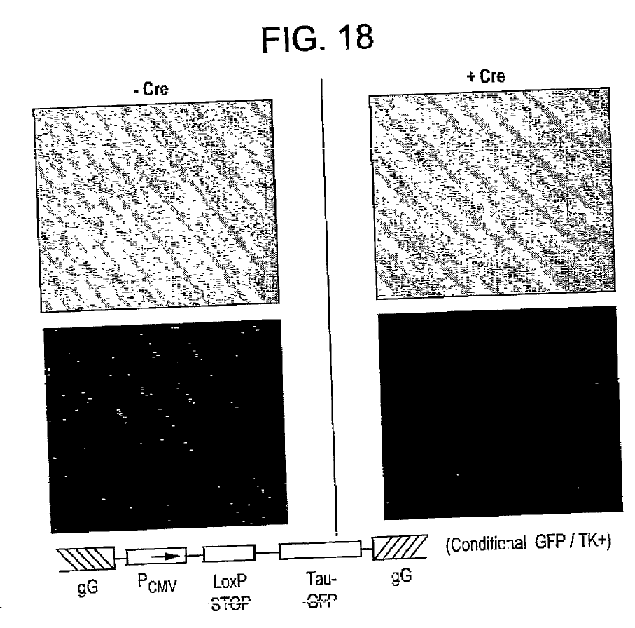

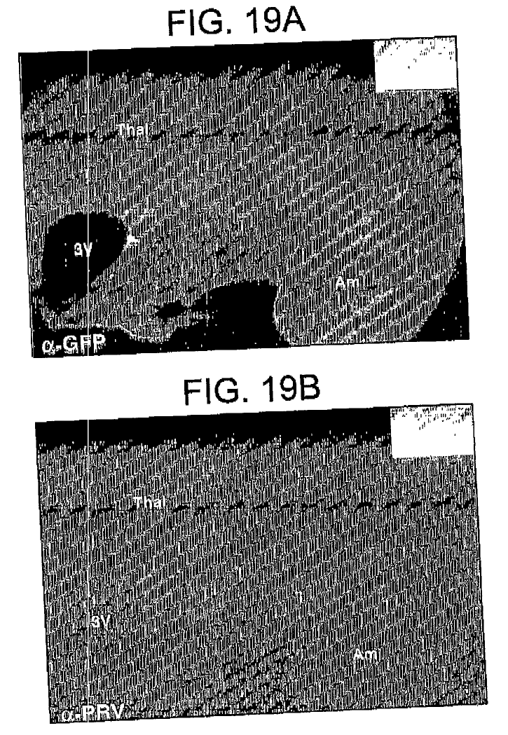

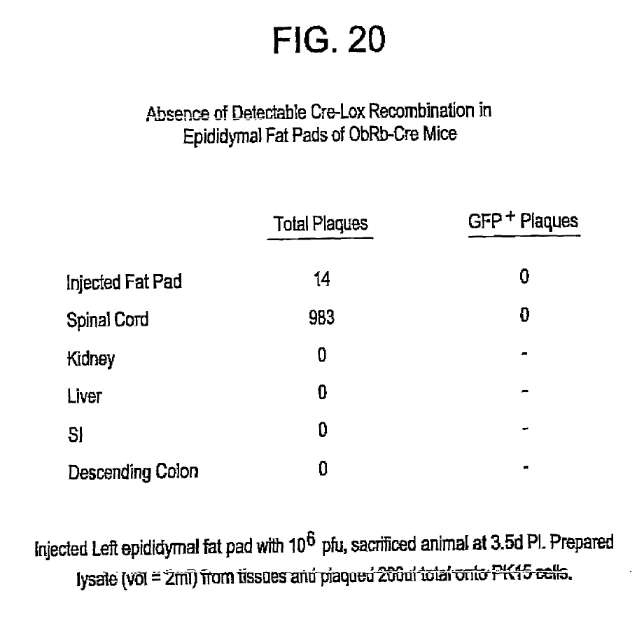

FIG. 12 depicts an alternative conditional lethal virus construct. Here the approach is similar to that described in FIG. 11; however, aDT expression is driven by a Gal4 promoter. The Gal4 promoter is activated by the VP16AD-Gal4DBD-hPG LBD fusion protein which is encoded by the first cistron in the bicistronic message. The presence of the mutated human progesterone (hPG) ligand binding domain in the activator allows gene expression to be positively controlled by the progesterone receptor ligand RU486.

FIG. 13 Lox stem loop structure.

Illustrates how indirect repeats that make up the single loxP element that remains after recombination can lead to the formation of a stem-loop in the 5′ untranslated region of the transcript encoding Tau-GFP. Similar stem-loops in 5′ UTRs have been shown to impede translation initiation at nearby open reading frames. The formation of the stem-loop is a thermodynamically favored event.

FIG. 14 GFP expression in cells infected with Bartha TetOff-TK-GFP. The DNA cassette shown was cotransfected with Bartha-Blue nucleocapsid DNA into PK15 cells as described earlier. The resulting lysate was the plated onto PK15 cells that had been stably transfected with pcTet-Off (Tet-Off ORF subcloned into pcDNA3 (Clontech)) and the resulting viral plaques examined for GFP fluorescence. Shown is a GFP fluorescent plaque (left) alongside another non-fluorescent plaque. Southern analysis of the viral DNA from this plaque confirmed that this clone contained the conditional expression cassette.

FIG. 15 Cre-dependent expression of Tet-Off triggers GFP and TK synthesis. Following Cre mediated recombination, Tet-Off is produced which in turn stimulates expression of both TK and GFP through the bi-directional (Bi) promoter. The Bi promoter contains several Tet-Off binding elements.

FIG. 16 Screening for TetOff—GFP clone plaques on a cell line that constituitively expresses TetOff.

TetOff is expressed constituitively in a PK15 cell line that was stably transfected with pcTetOff. TetOff produced by this cell line is sufficient to activate expression in trans from promoters containing tet-responsive elements (TRE's) in both plasmids and viral genomes. In the scenario shown, the cell will only fluoresce when infected by a virus that contains the Bi-GFP cassette (or derivative thereof). Neither uninfected nor wild-type virus infected cells will express GFP.

FIG. 17 Sympathetic innervation of Fat Pad.

Shown is the neural circuit innervating adipose tissue. Dotted line represents indirect connections of the hypothalamus to the spinal cord, IML, intermedial lateral cell column.

FIG. 18 GFP expression by BaLT is dependent on Cre.

BaLT was grown on either PK-15 (left panels) of PK-Cre cells (right panels) and examined for GFP fluorescence. Below, diagram illustrating the GFP conditional expression cassette used to generate BaLT.

FIG. 19 GFP expression by BaLT following fat pad injection.

GFP expression is found in both the hypothalamus and amygdala (upper panel). These GFP expressing neurons are a subset of the total number of infected neurons as seen by ant-PRV immunostaining (bottom panel).

FIG. 20 Absence of GFP-expressing virus in spinal cords of ObRb Cre mice. 3.5 days following injection of BaLT into fat pads, lysates were prepared from spinal cords pf infected ObRb Cre mice. Lysates were examined for viral particles that would express GFP by plaquing onto PK15 tissue culture cells.

### DETAILED DESCRIPTION

One of the challenges facing neurobiologists is mapping the connections between neurons within the mammalian brain. Reagants currently available only allow determination of connections between gross brain regions, yielding little or no information regarding the connections made to specific neuron types. The present invention provides a method or approach to specifically address this deficiency whereby it is now possible to trace neural circuits involving neurons that express particular genes (i.e. those involved in the production a neurotransmitter/peptide and/or receptor).

Tracing of neural circuits in the CNS has been limited by the availability of suitable methodology for trans-synaptic tracing. The precise delineation of the neural circuit that responds to any factor or stimulus is a necessary first step toward a fuller understanding of the mechanisms responsible for the regulation of behavior. The present invention relates to the development of novel recombinant neurotropic viruses, particularly an alpha-herpesvirus (pseudorabies virus; PRV), that are capable of retrograde tracing of specific neural connections across multiple synapses. While PRV is known to be transmitted across multiple synapses, conventional tracing strains infect neurons indiscriminately.

Pseudorabies virus (PRV), an alphaherpesvirus, replicates preferentially in neurons and neuron to neuron transmission of the virus is predominantly, if not exclusively, trans-synaptic. Thus, PRV has been a useful tool in the gross characterization of neural connections of the CNS and PNS. The present invention provides a recombinant pseudorabies virus that can produce a productive infection only after infecting a neuron that expresses our gene of interest. In a particular embodiment, a cre/lox system is utilized in which a gene cassette inserted into a replication-defective virus permits cre-dependent expression of a marker protein, Tau-GFP, and thymidine kinase, which rescues the replication deficiency.

In order to trace the connections of specific classes of neurons, we constructed a PRV recombinant that is conditionally expressed, and is particularly dependent on a Cre-mediated recombination event for expression of the essential tk gene and green fluorescentprotein (GFP) (a marker peptide which can readily be assessed). This virus is inert in wild-type mice but becomes both replication competent and capable of expressing GFP when injected into synapsin-Cre transgenic mice. This virus and similar conditional neurotropic viruses of the present invention can be used for tracing any neural circuit in mice or any animal where transgenic strains can be constructed with any specific recombinase protein expressed in neurons.

The Bartha strain of PRV is an attenuated, live vaccine strain that is propagated retrogradely in chains of synaptically-connected neurons, and is utilized in particular examples provided herein. To construct a PRV-Bartha strain that was conditional for both replication and for expression of the Tau-GFP marker protein, a deletion was introduced into the endogenous thymidine kinase (tk) gene of a Bartha recombinant that carried an insertion of the lacZ gene in the dispensable gG locus (Ba-Blu). A bicistronic expression cassette was then constructed in which the CMV immediate early promoter transcribes a tau-GFP fusion gene followed by an internal ribosome entry site (IRES) and the herpes simplex virus tk gene. In addition, Lox-STOP cassette was inserted between the CMV promoter and the start of the Tau-GFP open reading frame. This Lox-Stop cassette consists of two LoxP elements flanking three separate polyadenylation signals which function to prevent expression of sequences downstream of the STOP cassette. As a result, expression of the bicistronic tau-GFP/IRES/tk transcript is conditional on a Cre-mediated recombination event to excise the STOP cassette. The lacZ insert in Ba-Blu was then replaced by this construct by homologous recombination. The desired recombinant virus was identified by loss of B-galactosidase activity and further characterized by Southern blot analysis. The virus was named Ba2001.

In principle, the removal of the Lox-STOP cassette by Cre recombinase should activate the expression of both the tau-GFP and tk genes. This modification to the viral genome is permanent. Therefore, following infection of a neuron that expresses Cre, the STOP signals are removed and the resulting virus is capable of replicating in non-mitotic neurons because it expresses TK. In addition, the resulting virus is now capable of infecting second-, third-, etc: order neurons that project to the Cre-expressing neuron. These neurons are also marked by GFP expression.

Conditional expression of GFP in Ba2001, was confirmed by infecting wild-type PK15 and Cre-expressing TE-Cre cells. GFP fluorescence was not observed in cells that did not express Cre while cells expressing Cre infected with Ba2001 showed strong GFP fluorescence. Replication of Ba2001 in these cells was blocked by the pyrimidine arabinoside Ara-T which indicates that the herpes simplex tk gene was expressed.

Viral replication and GFP expression was also dependent on a Cre-mediated recombination in vivo. Here either C57B1/6 or C57B1/6 Synapsin-Cre transgenic mice received intracranial injections of Ba2001. Synapsin-Cre transgenic mice contain a Cre open reading frame under the control of the neuron-specific synapsin promoter resulting in Cre expression in all neurons. At five days post-infection, significant GFP fluorescence was observed in the cortex of Cre-expressing mice while none was evident in the wild-type mice. In addition, GFP-expressing neurons also expressed the late viral gC protein as demonstrated by anti-gC immunostaining. gC is expressed only after viral DNA replication. Neither GFP nor gC was expressed in wild-type mice. Synapsin-Cre transgenic animals showed signs of advanced infection while wild-type animals were completely asymptomatic. In order to test directly for conditional viral replication in vivo, viral particles were recovered from the brains of infected mice and scored the number of GFP fluorescent plaques following infection of cultured cells. Separate groups of wild-type and Synapsin-Cre animals received a single injection of 2.4×10⁶ pfu into the striatum and were sacrificed at 7 days post-infection. Lysates were prepared from the contralateral striatum from each brain and used to infect monolayer tissue culture cells. Plaques were grown, scored for GFP fluorence and counted. Lysates from infected Synapsin-Cre mice produced large number of fluorescent plaques, indicating that the virus had undergone Cre-mediated recombination in vivo. This was not the case in the infected wild-type mice where only a small number of non-GFP expressing plaques were obtained, presumably resulting from residual inoculum. GFP positive plaques were never recovered from wild-type mice. These results indicate that Ba2001 is dependent on a Cre-mediated recombination event for both replication and GFP expression.

The conditional method was tested in a specific model for leptin regulation. The recombinant virus was injected into the arcuate nucleus of the hypothalamus of separate strains of mice that express Cre in either neuropeptide Y (NPY) positive or leptin receptor (ObR) positive neurons. Both classes of neurons play an important role in regulating feeding behavior. Sectioning of brains at different times after injection identified a series of CNS neurons that project to the NPY or ObR neurons through one or more synapses. These studies identified inputs to NPY and ObR positive neurons from several hypothalamic nuclei as well as the amygdala, thalamus, the pyriform and somatosensory cortex and hippocampus. Our data indicate that in addition to leptin, signals from several higher cortical structures are integrated by groups of hypothalamic neurons regulating weight. This provides the first direct evidence that higher centers may modulate leptin signaling in the hypothalamus. Studies of the neurochemical nature of these inputs are likely to have important implications for understanding the ways in which this complex behavior is regulated. In a particular embodiment of this strategy, the recombinant virus, named Ba2001, infects transgenic or recombinant mice that co-express Cre recombinase with the gene-of-interest. This modification by cre is permanent. Following infection of a neuron that expresses cre recombinase, the virus is modified and begins expressing thymidine kinase, allowing DNA replication and the production of infectious virus. These newly produced virus will then infect any neurons that synapse on (make a connection to) the cre expressing neuron. Furthermore, once modified by cre recombinase, the infected cell also expresses GFP, which allows easy visualization of the cre expressing neuron and any neurons that subsequently become infected by this modified virus. In summary, the recombinant strain of PRV provided herein permits the identification and assessment of circuits that contain specific classes of neurons.

The invention provides a method for characterizing neural circuits and neural connections. The invention provides a method for tracing of neural circuits or neural connections across synapses.

The present invention provides a method for characterizing a neuronal pathway in an animal which utilizes a neurotropic virus capable of spreading between synaptically connected neurons which is conditional for marker peptide expression, whereby said marker peptide is expressed upon a specific recombination event and/or in the presence of an inductive agent. In the method, the neurotropic virus is introduced, followed by introduction or activation of a recombinase or inductive agent, such that the marker peptide is expressed and can be monitored. In as much as the neurotropic virus spreads between synaptically connected neurons and its presence and/or replication is indicated by the marker peptide, the neural circuit or neural connections across synapses can be characterized by assessing or monitoring marker peptide expression.

The invention provides a method for characterizing a neuronal pathway in an animal comprising:

a) introducing a neurotropic alphaherpesvirus capable of spreading between synaptically connected neurons which is conditional for marker peptide expression, whereby said marker peptide is expressed upon a specific recombination event;

b) introducing or activating a recombinase such that said recombination event is induced and marker peptide expression occurs; and

c) monitoring marker peptide expression.

The method of the present invention contemplates utilizing a neurotropic alphaherpesvirus which is also conditional for replication, whereby replication is permissive upon a specific recombination event, further comprising:

a) introducing or activating a recombinase such that said recombination event is induced and replication of said neurotropic virus occurs.

In one contemplated method of the present invention, both marker protein expression and replication are conditional upon the same specific recombination event.

The invention further provides a method for characterizing a neuronal pathway in an animal comprising:

a) introducing a neurotropic alphaherpesvirus capable of spreading between synaptically connected neurons which is conditional for marker peptide expression, whereby said marker peptide is expressed in the presence of an inductive agent;

b) introducing said inductive agent such that marker peptide expression occurs; and

b) monitoring marker peptide expression.

In a variation on this method, the neurotropic alphaherpesvirus is also conditional for replication, whereby replication is permissive upon a specific recombination event, and the method includes introducing a recombinase such that said recombination event is induced and replication of said neurotropic virus occurs.

Site-specific recombinases mediate DNA rearrangements at specific target sites and can be classified into several distinct families on the basis of their amino acid sequences and certain features of their reaction mechanisms. The integrase family of recombinases includes λ integrase from bacteriophage λ and Cre recombinase from bacteriophage P1 (Stark, W. M. et al (1992) Trends Genetics 8, 432-439). Another subfamily of the integrase family includes yeast recombinases, which include the FLP recombinase encoded on the yeast 2μ circle from S. cerevisiae (Sadowski, P. D. (1995) Prog Nucl Acids Res Mol Biol 51, 53-91), the R recombinase from Zygosaccharomyces rouxii (Araki, H. et al (1992) J Mol Biol 225, 250-37), and the Kw recombinase from Kluyveromyces waltii (Ringrose, L. et al (1997) Eur J Biochem 248, 903-912).

The method of the present invention may utilize a recombinase from prokaryotic or eukaryotic cells. The invention particularly provides for the use of a recombinase selected from the group of Cre, FLP, λ integrase, R recombinase or Kw recombinase.

The method of the invention includes methods wherein the expression of the recombinase is under the control of a heterologous expression control sequence. The heterologous expression control sequence may be expressed in particular cell types, may respond to particular inductive agents, or may be constitutively expressed.

In a particular method of the present invention the heterologous expression control sequence controlling the recombinase is neural cell specific. In a further particular method of the present invention, the heterologous expression control sequence is that of a neuropeptide or neuroactive peptide. The heterologous expression control sequence for use in the method of the invention may be selected from the group of NPY, Ob-R, POMC, MC4R, MCH and AgRP.

In the method for characterizing a neuronal pathway of the present invention, a neurotropic virus is introduced to an animal, which virus is conditional for marker peptide expression. The marker peptide is conditionally expressed upon a specific recombination event and/or in the presence of an inductive agent. In the method, the neurotropic virus is introduced, followed by introduction or activation of a recombinase or inductive agent, such that the marker peptide is expressed and can be monitored. The marker peptide may be monitored through various means or techniques and tools available to the skilled artisan, including by recognition as a fluorescent dye or compound (as in, for example, GFP and the various fluorescence forms of GFP), by antibody recognizing the protein, by recognition or assessment of protein expression using RT-PCR, etc., and by a cellular effect of the marker peptide (for example as in a peptide toxin, apoptotic protein, etc.). The cellular effect of the marker peptide may include but not be limited to cell death in the infected cell, cessation of viral infection in the infected cell, activation of cellular genes in the infected cell.

Conditional expression may be controlled by the introduction or expression of an inductive agent. An inductive agent may act to stimulate or turn on expression of an essential viral gene, or of the marker peptide. Nonlimiting examples of inductive agents include but are not limited to essential viral proteins, promoter activators (including, for instance, antibiotics (e.g. tetracycline, doxycycline), hormones, soluble factors, cell factors, transcription factors, receptor ligands, etc.

In the method of the present invention, the neurotropic alphaherpesvirus may be introduced by intranasal infection. In a method of the present invention the alphaherpesvirus may be introduced by ocular infection or ocular injection. In a further method of the invention, the neurotropic alphaherpesvirus may be introduced by injection into the brain of said animal. In addition, or alternatively, the neurotropic alphaherpesvirus may introduced by injection or infusion into the spinal cord of said animal. Still further contemplated are methods wherein the neurotropic alphaherpesvirus is introduced by injection into a peripheral organ of said animal.

The invention also relates to recombinant neurotropic viruses which are capable of tracing of neural connections across multiple synapses. The neurotropic viruses of the present invention, particularly neurotropic alphaherpesviruses, are capable of conditionally expressing a marker peptide. The neurotropic virus conditionally expresses the marker peptide upon a specific recombination event and/or in the presence of an inductive agent.

The invention provides a recombinant neurotropic alphaherpesvirus capable of conditionally expressing a marker peptide, wherein said marker peptide is expressed upon a specific recombination event and/or in the presence of an inductive agent.

The neurotropic virus of the present invention includes a pseudorabies virus.

The neurotropic alphaherpesvirus of the present invention can be derived from any strain of PRV, and may particularly derived from a PRV strain selected from teh group of Bartha, Becker or Kaplan. The present invention particularly provides a neurotropic alphaherpesvirus which is selected from the group of Ba2000 or Ba2001.

The invention further provides a transgenic animal harboring a neurotropic alphaherpesvirus capable of spreading between synaptically connected neurons which is conditional for marker peptide expression.

The invention also includes a transgenic animal harboring a neurotropic virus which is conditional for marker peptide expression and which is also conditional for replication.

The invention further provides a transgenic animal suitable for infection by a conditionally lethal herpesvirus, wherein on infection of the transgenic animal, the herpesvirus may be conditionally activated and thereby the transynaptic path of viral infection may be assessed. In addition to assessment of the neural pathway via viral infection, the conditional activation of viral infection in the transgenic animal may be utilized to assess the function of a specific neural pathway (for instance by infection in a specific region of the brain or by infection via a specific peripheral organ or peripheral location) via physiological effects, behavioral effects or other phenotypic assessment of the transgenic animal. The transgenic animal of the present invention may express (conditionally or unconditionally) a recombinase or other inductive agent necessary for conditional induction of the virus or for conditional lethality or cell death of the virus-infected neurons.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and -35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into MRNA, which is then translated into the protein encoded by the coding sequence.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stablely transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

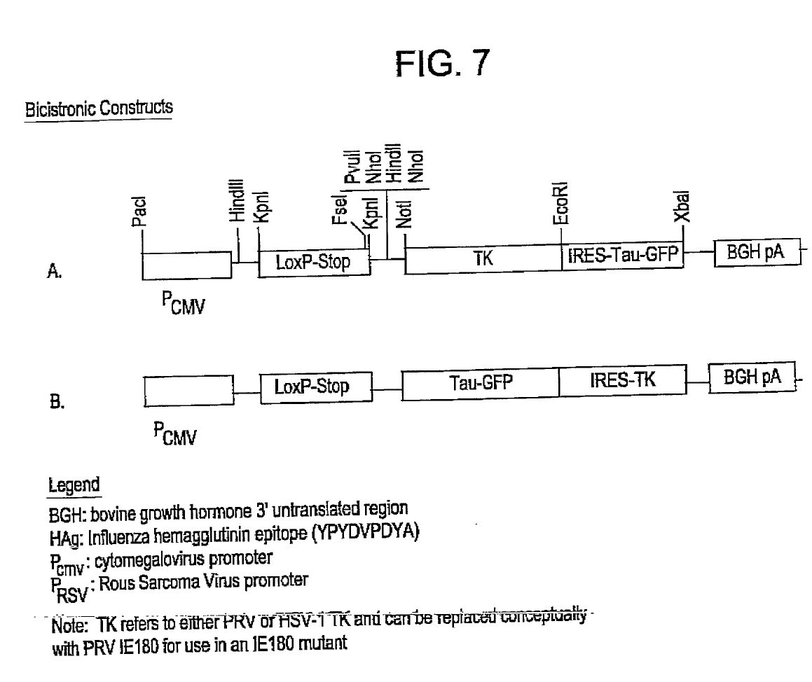

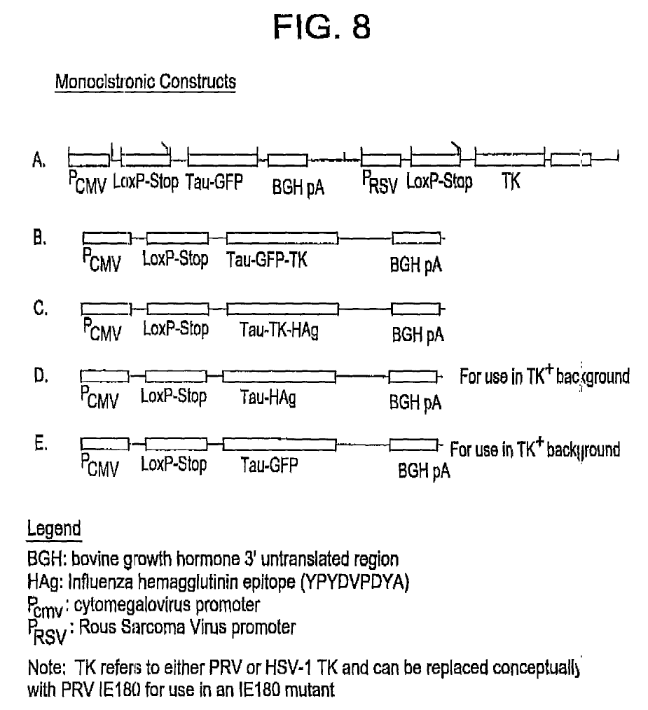

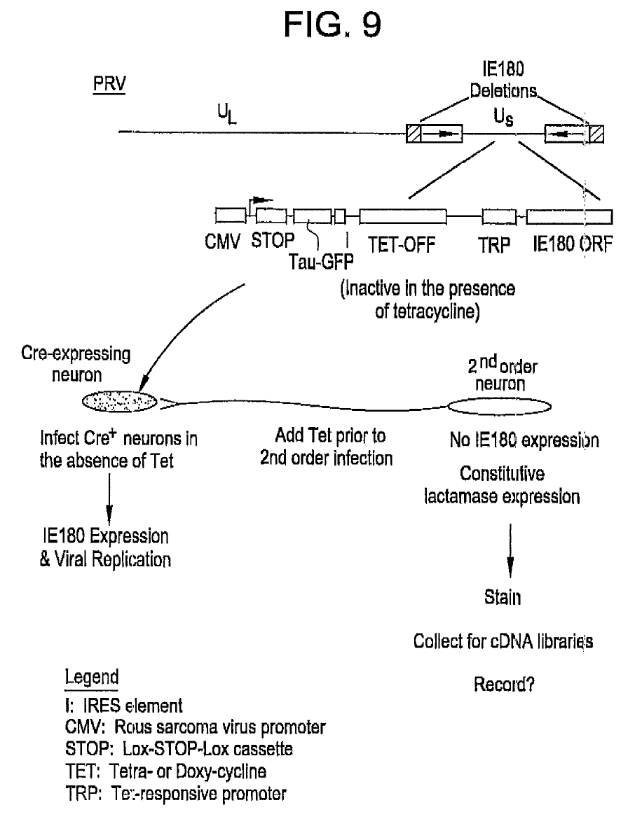

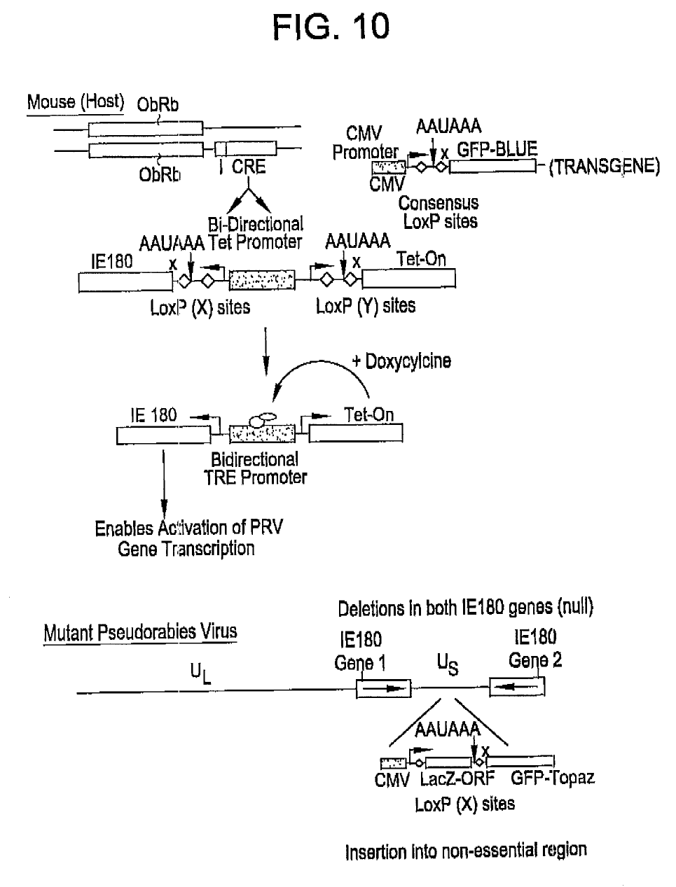

The use of neurotropic viruses, including PRV, to trace and characterize neuronal pathways can be extended and varied in a number of other ways. Examples of additional conditional expression schemes and PRV constructs to be utilized, including bicistronic and monocistronic constructs are provided in FIGS. 7-10. Neurotropic viruses may be utilized that substitute different GFP variants, which are distinguishable with the appropriate fluorescence filters (28). In this way, the spatial organization of neurons in nuclei that receive inputs from a number of distinct sites could be mapped both in that nucleus and at the level of the inputs to that nucleus. The extent of overlap of pathways would be identifiable by double labeling.

Alternative site-specific recombinases (e.g., FLP, λ integrase, R recombinase, Kw recombinase) may be utilized to construct a distinct series of recombinase-dependent viruses. By utilizing a virus expressing a GFP protein that was distinguishable from that of Ba2001 along with Ba2001, it would be possible to trace simultaneously different pathways in animals that express Cre in some neurons and Flp in others.

While the Bartha strain utilized in Example 1 spreads predominantly by retrograde transport, less virulent derivatives of the Becker strain can spread both retrograde and anterograde. Thus the creation of analogous PRV strains on the Becker background could make it possible to also trace the efferent connections of these (and other) neurons. By labeling a Becker construct with a distinct GFP or other marker (e.g. epitope tag marker) and utilizing it in concert with a retrograde virus construct (e.g. Ba2001), the double labeled pathway would mark retrograde tracing, while the singly Becker labeled tracing would mark anterograde.

PRV viruses, or other herpesviruses, can be constructed that carry mutations in viral genes that regulate expression of host RNAs. For example, UL41 is the PRV homologue of the herpes simplex virus virion host shut off gene (vhs) whose function is thought to be to degrade mRNA during infection (29,30). In the absence of vhs function, host genes are continually expressed on infection of a UL41 or other vhs equivalent mutant. Infection of Cre expressing animals with a UL41 mutant would therefore make it possible to determine the neuron's transcriptional profile using DNA microarrays, particularly facilitated by the expression of a marker peptide, for instance a fluorescent label or epitope tag, which identifies the PRV infected cell(s). It would also be possible, by virtue of the fluorescent label or epitope tag marker, to sort (e.e., FACs sort) and thereby isolate the virus-infected cells for transcriptional profile analysis.

Design and utilization of a conditionally lethal virus provides the ability to specifically ablate or disrupt cells in a neural pathway. These viruses conditionally express a toxin, apoptotic protein or other agent that terminates the virus life cycle and/or causes cell death in the virus infected cell. This system results in the destruction of a portion of the neural circuit. Analysis of the resulting phenotype of an animal infected with such a conditionally lethal virus would provide insight regarding the function of the affected neurons. These animals may provide unique models for conditions, particularly neural conditions, wherein a specific neural circuit or response pathway is altered or obliterated. Examples of vectors for use as a conditionally lethal system are depicted in FIGS. 11 and 12.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

As used herein, “pg” means picogram, “ng” means nanogram, “ug” or “μg” mean microgram, “mg” means milligram, “ul” or “μl” mean microliter, “ml” means milliliter, “l” means liter.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast α-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence/host combinations that will express the DNA sequences of this invention on fermentation or in large scale animal culture. The present invention also relates to a variety of diagnostic applications, including methods for detecting the presence of peptides, such as marker peptides, inductive agents, and induced polypeptides, including polypeptide ligands. The marker peptides or inductive agents or induced polypeptides can be used to produce antibodies to itself by a variety of known techniques, and such antibodies could then be isolated and utilized as in tests for the presence of marker peptides or inductive agents or induced polypeptides the in suspect target cells.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890.

The presence of marker peptides or inductive agents or induced polypeptides in cells can be ascertained by the usual immunological procedures applicable to such determinations. A number of useful procedures are known. Three such procedures which are especially useful utilize either the marker peptides or inductive agents or induced polypeptides labeled with a detectable label, antibody Ab₁ labeled with a detectable label, or antibody Ab₂ labeled with a detectable label. The procedures may be summarized by the following equations marker peptides or inductive agents or induced polypeptides:

A.˜* +Ab₁=˜*Ab₁

B.˜+Ab*=˜Ab₁*

C.˜+Ab₁+Ab₂*=˜Ab₁Ab₂*

The procedures and their application are all familiar to those skilled in the art and accordingly may be utilized within the scope of the present invention. The “competitive” procedure, Procedure A, is described in U.S. Pat. Nos. 3,654,090 and 3,850,752. Procedure C, the “sandwich” procedure, is described in U.S. Pat. Nos. RE 31,006 and 4,016,043. Still other procedures are known such as the “double antibody,” or “DASP” procedure.

It will be seen from the above, that a characteristic property of Ab₂ is that it will react with Ab₁. This is because Ab₁ raised in one mammalian species has been used in another species as an antigen to raise the antibody Ab₂. For example, Ab₂ may be raised in goats using rabbit antibodies as antigens. Ab₂ therefore would be anti-rabbit antibody raised in goats. For purposes of this description and claims, Ab₁ will be referred to as a primary or anti-peptide/agent antibody, and Ab₂ will be referred to as a secondary or anti-Ab₁ antibody.

The labels most commonly employed for these studies are radioactive elements, enzymes, chemicals which fluoresce when exposed to ultraviolet light, and others.

A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate.

The marker peptides or inductive agents or induced polypeptides or its binding partner(s) can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLE 1

### Neural Tracing With a Conditional Herpesvirus: Mapping of Inputs to a Feeding Center in the Hypothalamus

Abstract

Feeding is a complex behavior that is modulated by the integrated effects of a number of nutritional, metabolic and hormonal inputs. The precise delineation of the neural circuit that responds to these inputs is a necessary first step toward a fuller understanding of the regulation of this behavior. Here we report the development of a novel recombinant herpesvirus pseudorabies virus (PRV) that can be used for retrograde tracing of specific neural connections across multiple synapses. While it has been previously shown that in the CNS wild type PRV is transmitted exclusively across synapses, wild type viral strains infect neurons indiscriminately. In order to trace the connections of specific classes of neurons, we developed a recombinant PRV that is activated in a cell specific manner and can be used for retrograde tracing from selected neurons. The recombinant PRV is derived from the Bartha strain, which has been shown to be transmitted in a retrograde fashion. This virus encodes a green fluorescent protein (GFP) marker and replicates only in neurons that express the Cre recombinase and in neurons in synaptic contact with the originally infected cells. The virus was injected into the arcuate nucleus of mice that express Cre only in those neurons that express neuropeptide Y (NPY) or the leptin receptor. Sectioning of the brains revealed that these neurons receive inputs from neurons in other regions of the hypothalamus as well as the amygdala, cortex and other brain regions. These data suggest that higher cortical centers modulate leptin signaling in the hypothalamus.

This method of neural tracing may prove useful in studies of other complex neural circuits. The recombinant PRV virus is dependent on a Cre mediated recombination event for expression of the essential thymidine kinase (TK) gene and green fluorescent protein (GFP). In the absence of Cre expression in a wild type mouse, the virus does not express TK, an enzyme essential for virus replication in neurons. However when the virus infects neurons in SynI-Cre transgenic mice, TK is expressed along with GFP and spreads to all neurons in synaptic contact with the infected neurons. Syni-Cre mice express cre recombinase in all neurons.

This virus was injected into the brains of transgenic mouse strains that express cre in neurons that produce NPY, which is an abundant RNA, or neurons that express the leptin receptor, which is a low abundance transcript. NPY and ObRb expressing neurons in the hypothalamus are known to play an important role in regulating feeding behavior. Sectioning of brain after injection into the arcuate nucleus of the hypothalamus identified a series of CNS loci that project to these neurons through one or more synapses. These studies identified inputs to these neurons from several other hypothalamic nuclei as well as the amygdala, the pyriform, entorhinal, and retrosplenial cortex and parabrachial nucleus. These data indicate that in addition to leptin, signals from several higher cortical structures are integrated by groups of hypothalamic neurons regulating weight. These data represent the first direct evidence that higher centers may modulate leptin signaling in the hypothalamus.

The use of this viral vector for neural tracing has several useful features relative to other methods: i) the tracing studies can be limited to discrete regions by injecting the virus sterotaxically; ii) the system allows tracing of multiple order neurons using even low abundance transcripts to drive cre synthesis; iii) the use of the Bartha substrain allows retrograde tracing while another viral strain, the Becker strain, can be used for anterograde tracing; iv) high level expression of the marker protein is evident in all infected neurons allowing a detailed morphologic analysis v) A virtually unlimited number of markers can be inserted into the virus making it possible to simultaneously trace multiple pathways. This new methodology may be of use in other studies of complex neural circuits.

Introduction

The decision to initiate feeding is dependent on a variety of motivational, metabolic and hormonal factors including the plasma level of leptin (1). While higher cortical centers can influence food intake, the precise nature of the neural connections by which such influences are effected is largely unknown. Specifically it is not known whether higher cortical signals, for example the conscious desire of some to lose weight, project to hypothalamic centers that regulate food intake and weight in response to leptin, glucose and other signals.

Leptin is an adipocyte-derived hormone that plays an important role in regulating feeding (2). Changes in plasma leptin concentration have been shown to alter food intake in rodents. Thus leptin deficiency results in increased food intake while administration of exogenous leptin reduces food intake. Leptin's effects on food intake are the result of its direct effect on distinct classes neurons that are distributed among several hypothalamic nuclei including the arcuate nucleus (ARC), the ventromedial (VMH), lateral (LH) and dorsomedial (DMH) nuclei (3). While neurons in these nuclei are known to receive inputs from other brain regions, the precise nature of the inputs to neurons expressing the leptin receptor are not known. The identification of such neural inputs requires that one be able to trace neural connections across one or more synapses.

The α-herpesvirus pseudorabies virus (PRV) has been previously used for tracing neural circuits across multiple synapses (4). After injection of PRV into a number of peripheral sites including the heart, gastrointestional (GI) tract and liver, viral antigens can be detected in precisely those brain regions known to innervate these organs. PRV has also been used to be traced polysynaptic circuits following direct injection into the central nervous system (CNS) (5). However, because the virus infected many different neurons, no information could be deduced regarding inputs to specific classes of neurons; for example, those expressing a particular neurotransmitter, neuropeptide or receptor.

Here we report the development of a recombinant PRV that is dependent on a Cre-mediated recombination event for replication and for expression of green fluorescent protein (GFP). This virus is only activated in Cre expressing cells and was used to map CNS inputs to hypothalamic neurons that play a role in regulating food intake.

Results

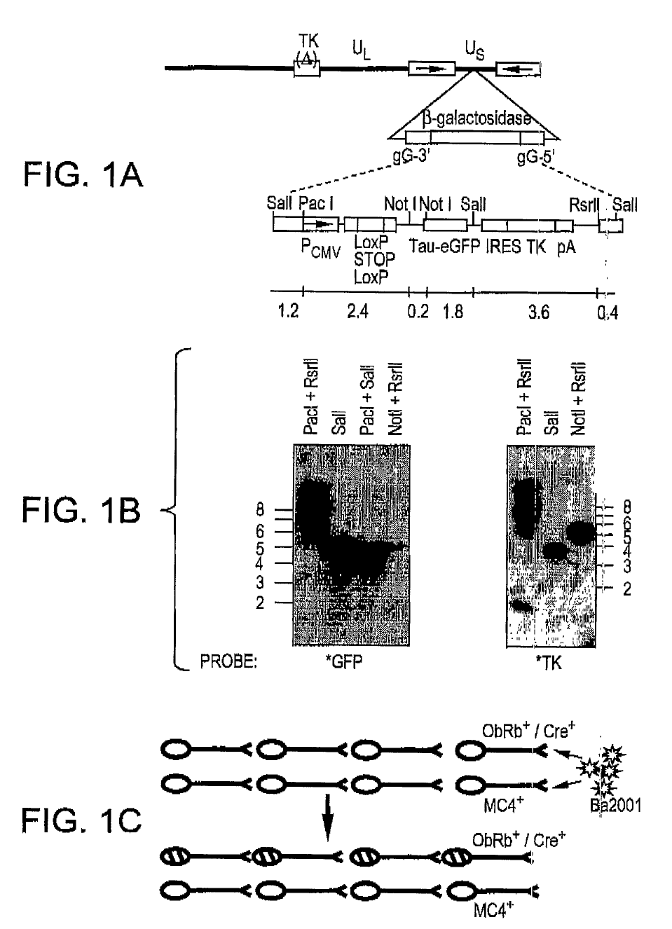

The Bartha strain of PRV is an attenuated live vaccine strain that is propagated primarily in a retrograde fashion (i.e.; opposite to the direction of impulse transmission) along chains of synaptically connected neurons. We constructed a PRV-Bartha strain that was conditional both for replication (in non-mitotic cells) and for expression of the Tau-GFP marker. We first introduced a deletion into the endogenous thymidine kinase (TK) gene of a recombinant PRV that already carried an insertion of the lacZ gene in the dispensable gG locus (TK-BaBlu) (FIG. 1A) (6). We next constructed a bicistronic expression cassette in which the cytomegalovirus (CMV) immediate early promoter was cloned upstream of a Tau-GFP fusion gene followed by an internal ribosome entry site (IRES) immediately 5′ to the herpes simplex TK gene . A Lox-STOP-Lox cassette was inserted between the CMV promoter and the start of the Tau-GFP open reading frame. This STOP cassette, which is comprised of two LoxP elements flanking an SV40 polyadenylation signal and 5′ splice donor site, prevents expression of downstream sequences unless it is removed by Cre-mediated recombination. (7). Thus, expression of both TK and GFP is conditional on a Cre-mediated recombination event. This construct was then used to replace the lacZ insert in the TK-BlaBlu virus by homologous recombination. The recombinant virus, which we named Ba2001, was initially identified by loss of B-galactosidase (B-gal) activity and further characterized by Southern blot analysis (FIG. 1B) (8).

Since TK is required for viral DNA synthesis in neurons, Ba2001 would not be expected to replicate in neurons that do not express Cre recombinase (9). Infection of a neuron that expresses Cre would be expected to lead to the removal of the STOP signals, thereby rendering the virus capable of replicating in other neurons in synaptic contact with the original neuron. These infected neurons should also express GFP. The removal of the STOP signals is permanent, allowing the subsequent retrograde infection and labeling of any afferent neurons whether or not they express Cre.

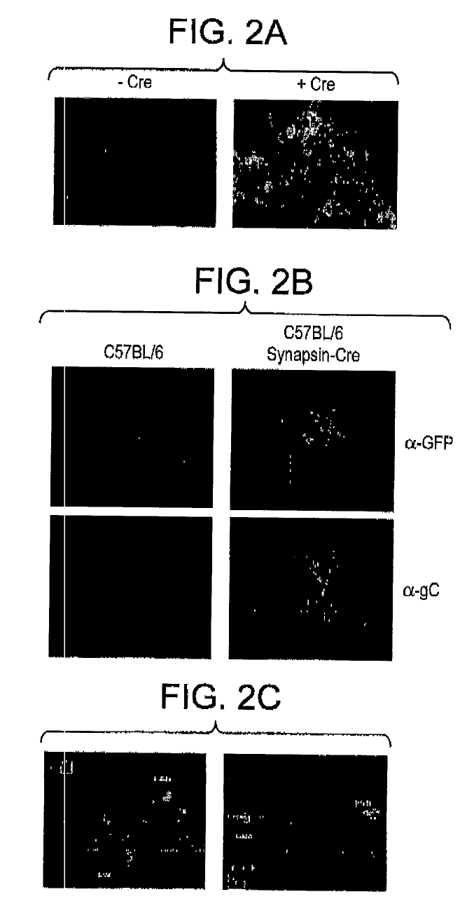

Conditional expression of GFP in Ba2001 was confirmed by infecting untransfected PK15 and Cre-expressing TE-Cre cells (10). GFP fluorescence was never observed in Ba2001 infected cells that did not express Cre but was readily detectable in infected Cre-expressing cells (FIG. 2A). Replication of Ba2001 in TE-Cre cells was blocked by the pyrimidine arabinoside Ara-T (11). This confirms that the herpes TK gene was expressed (data not shown).

Viral replication and GFP expression in vivo were also dependent on Cre-mediated recombination (FIG. 2B). Ba2001 was injected intracranially into wild type C57B1/6 mice and C57BI/6 TG SynI-Cre mice (12). SynI-Cre mice express Cre under the control of the neuron-specific synapsin promoter resulting in Cre expression in all neurons. At five days post-infection, significant GFP fluorescence was observed in the cortex of Cre-expressing mice but not in the wild-type mice (FIG. 2B). The GFP expressing neurons also stained for the gC protein, a late viral protein, expressed after viral DNA replication (13). Neither GFP nor gC was expressed in wild type mice. SynI-Cre animals also showed signs of advanced infection and died while C57B1/6 mice were completely asymptomatic (data not shown).

To demonstrate that the STOP cassette had been excised in vivo, we rejected 2.4×10⁶ plague forming units (pfu) of Ba2001 into the striatum of wild type and SynI-Cre mice and sacrificed the animals 7 days later. Lysates were prepared from the contralateral striatum of each animal and used to infect PK15 cells in culture. Individual virus plaques were counted and scored for GFP fluorescence. We recovered 75 fluorescent plaques out of a total of 76 plaques/microliter from the lysates of infected SynI-Cre mice and no fluorescent plaques (0/7) from infected wild type mice (TABLE 1). This indicates that the virus had undergone Cre-mediated recombination in vivo. Southern Blots confirmed that the LOX cassette had been removed in the fluorescent viruses.

TABLE 1
Recovery of Cre-Promoted Recombinant Virus from Infected Brains

| | Total Number of | Number of |
|---|---|---|
| Host | Plaques | Green Plaques |
| C57BL/6 | 7 | 0 |
| C57BL/6 | 76 | 75 |
| Synapsin-Cre | | |
| Cre-dependent rescue of viral replication in vivo. Ba2001 was injected | | |
| # into the striatum of wild type or Synl-cre mice. Lysates from the contralateral striatum | | |
| # were diluted and used to infect PK15 tissue culture cells. Plaques were grown, counted and | | |
| # scored for GFP fluorescence. GFP expressing virus was present exclusively in the lysates prepared from the brains of Synl-Cre mice. | | |

In order to confirm that the recombinant virus could trace known neural connections with fidelity, we infected the eye. The afferent connections to the eye have been well characterized using other methods (14, 15). Intravitreal injections of Ba2001 into CMV-Cre transgenic mice were performed. At 5 days post infection, GFP-expressing neurons were identified in the periaqueductal grey (PAG) region (FIG. 2C, left panel), the paraventricular nucleus of the hypothalamus (PVN) and the basomedial nucleus of the amygdala (BMA). These results are consistent with other tracing studies which have shown that neurons from the amygdala, PAG and PVN project to the intermediolateral cell column (ML) that in turn projects to the superior cervical ganglion (SCG) (15,16). The SCG in turn projects to the eye. The labeled neurons seen in the Edinger-Westphal nucleus are indicative of an ocularmotor nerve infection, a result typical of intravitreal infections with the Bartha strain. These data confirm that Ba20001 can be used to trace known pathways and further indicate that third order neurons and beyond can be visualized.

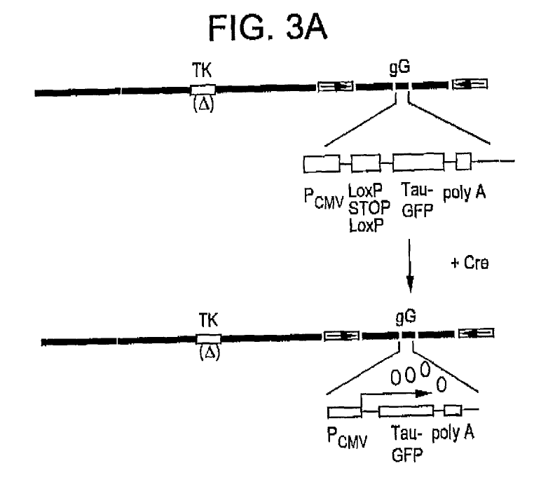

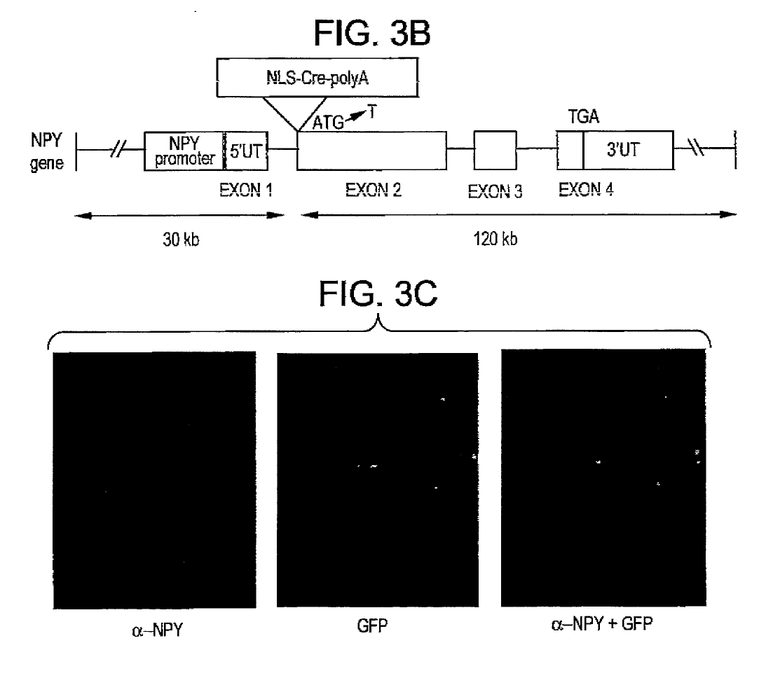

We next used Ba2001 to trace the connections from neurons in which Cre expression was restricted to neurons that express the neuropeptide Y (NPY) gene. NPY is a 36 amino acid peptide that increases food intake and body weight following intracranial and intra-hypothalamic injections (17). NPY is abundantly expressed in neurons in a large number brain regions including the cortex, medulla, olfactory bulb and the hypothalamus. Hypothalamic neurons that express NPY also co-expres the leptin receptor and are believed to play a key role in regulating feeding behavior (18). In these experiments, we used a bacterial artificial chromosome (BAC) transgenic mouse line that carries a modified 150 kB NPY BAC in which the Cre recombinase gene was inserted by homologous recombination into the ATG of the first exon of the NPY gene (FIG. 3B). In the resulting BAC, 30 kB of 5′ upstream sequence and 90 KB of sequences 3′ to the NPY gene flank a Cre-Poly A cassette. In order to confirm that the recombination event occurred only in NPY expressing cells, a TK⁻ virus conditional only for GFP expression was constructed (FIG. 3A). Since this virus is defective for DNA replication, only the primarily infected neurons were expected to express GFP (as well as immediate early and early genes). Thus expression of Tau-GFP should be restricted to those neurons that express NPY and Cre. After cortical injections of the virus into NPY-cre mice, large numbers of NPY-expressing cell bodies (scored using a specific anti-NPY antibody) were observed proximal to the injection site (FIG. 3C, left panel). GFP fluorescence was evident only in a subset of these neurons (FIG. 3C, middle panel). Superimposition of the anti-NPY and GFP images revealed yellow and red, but not green cells (FIG. 3C, right panel). If GFP expression were independent of Cre, green cells would have been observed. These data confirmed that GFP expression was restricted to Cre- and NPY-expressing neurons.

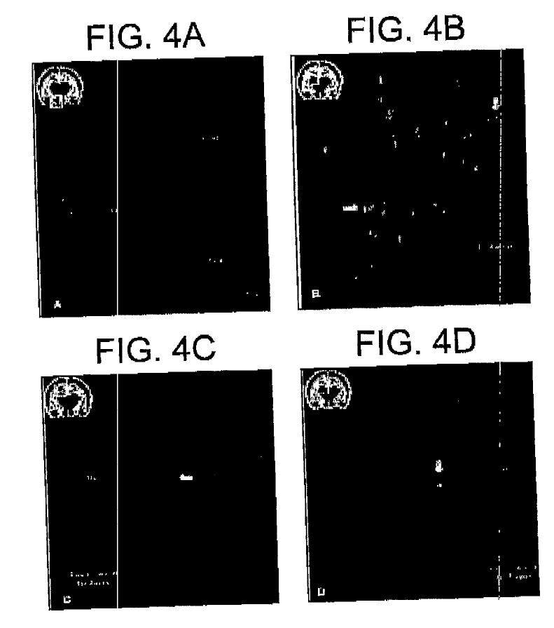

To map neural inputs to NPY expressing cells that play a role in regulating feeding behavior, Ba2001 was stereotaxically injected into the arcuate nucleus of the hypothalamus of NPY-Cre mice. The arcuate nucleus is the hypothalamic site with the highest level of NPY expression (19). Mice were sacrificed at various times post-infection and the pattern of expression of GFP was examined in serial sections of whole brains. At early times, GFP was expressed in several other hypothalamic nucleic, most notably the VMH and LH (FIG. 4A). This result confirms previous evidence that the arcuate nucleus receives inputs from these other hypothalamic nuclei (20). Retrograde infection by the virus from the hypothalamus was also observed in the medial dorsal nucleus of the thalamus (MD) (FIG. 4B) and the dentate gyrus (DG) (FIG. 4C). At 5 days, post-infection, the infection had spread to the piriform complex (Pir) and the ventral basolateral amygdala (BLV) (FIG. 4D). The later appearance of fluorescence at these sites suggests that they are most likely second or higher order neurons. A summary of the times at which each of the sites was first visualized and the intensity of fluorescence is shown in TABLE 2. At earlier stages of infection, GFP expression is restricted to hypothalamic nuclei. At 4 days post-infection, labeling is present in other sites including the amygdala, bed of stria terminalis (BST), and piriform cortex.

TABLE 2

NPY-Cre

| | |
|---|---|
| Time | ObRb-IRES-Cre |

| GFP Labeled Sites | Post-Infection | Frequency | Intensity | Frequency | Intensity |
|---|---|---|---|---|---|
| Arcuate Nucleus | 1.5 days | 2/2 | +++ | 4/4 | +++ |
| Ventromedial Hypothalamus | 3 | 3/3 | +++ | 5/5 | +++ |
| Dorsomedial Hypothalamus | 3 | 3/3 | ++ | 5/5 | +++ |
| Lateral Hypothalamus | 3 | 3/3 | ++ | 5/5 | ++ |
| Paraventricular Nucleus | 3 | 2/3 | + | 5/5 | ++ |
| Suprachiasmatic Nucleus | 3 | 0/2 | − | 3/5 | + |
| Amygdata | 4 | 2/2 | +++ | 4/4 | +++ |
| Bed of Stria Terminalls | 4 | 2/2 | +++ | 4/4 | ++ |
| Piriform Cortex | 4 | 1/2 (5 days) | + | 3/4 | + |
| Thalamus | 4 | 1/2 | + | 0/4 | − |
| Lateral Entorhinal Cortex | 5 | ND | − | 5/5 | ++ |
| Perirhinal Cortex | 5 | ND | − | 5/5 | ++ |
| Somatosensory Cortex 2 | 5 | ND | − | 3/5 | + |
| Mesencephalic Trigeminal Nucleus | 5 | ND | − | 1/5 | + |
| Somatosensory Cortex 1 | 7 | 0/2 | − | 3/3 | +++ |
| Retrosplenial Cortex | 7 | 0/2 | − | 3/3 | ++ |
| CA1/Hippocampus | 7 | 1/2 (4 days) | + | 1/3 | + |
| Lat. Paragigantacellular Nucl. | 7 | 0/2 | − | 1/3 | + |
| Raphe Magnus | 7 | 0/2 | − | 1/3 | + |

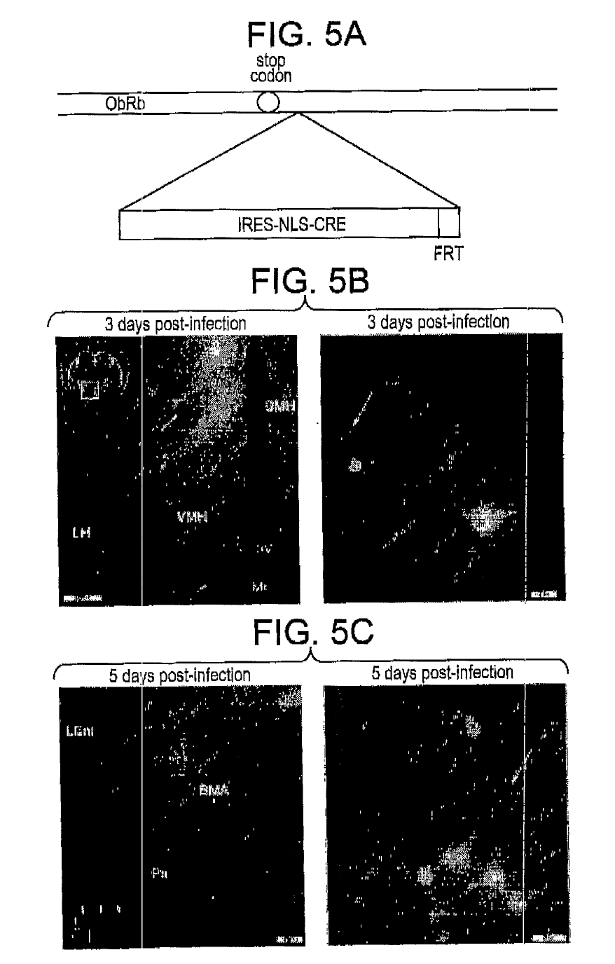

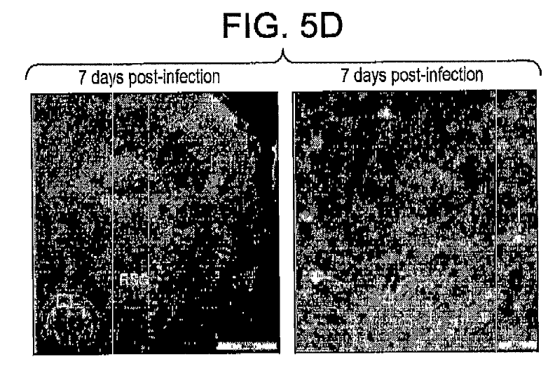

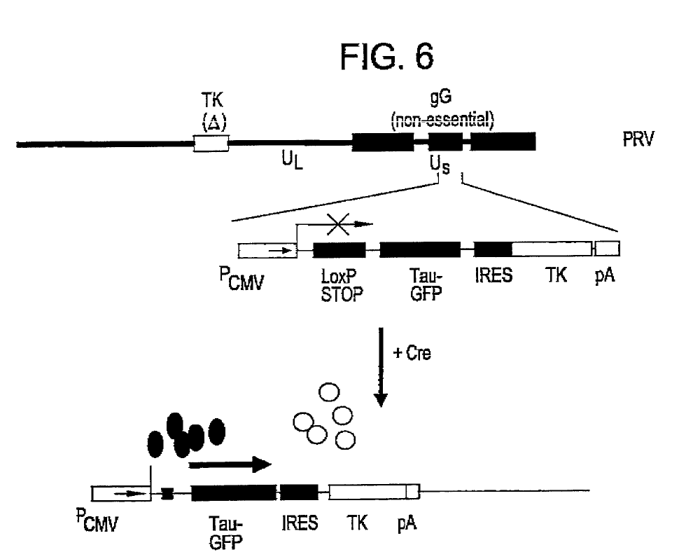

We also attempted to map the inputs to neurons expressing the leptin receptor (ObRb), which is much less abundant than NPY (FIG. 5A). The large size of the ObR gene precluded the use of the BAC transgenic approach so we introduced an IRES-Cre cassette by homologous recombination immediately 3′ to the ObRb stop codon (FIG. 5A). Productive infections were observed after injection of the virus in the hypothalamus but not after injection into the amygdala, pyriform cortex or cerebellum (data not shown). This is consistent with the known distribution of ObRb in the brain (21).

We next injected Ba2001 and fluorescent beads (to localize the injection site) into the arcuate nucleus of the Ob-R-Cre mice and prepared brain sections at various times after infection. A summary of the brain regions visualized after infection is shown in TABLE 2. As in the NPY experiments, the lag time between viral injection and GFP expression by the various neurons was used to tentatively assign a hierarchy among the connections. At 72 hours post-infection, GFP-expressing neurons were observed in the arcuate nucleus rostral to the injection site (FIG. 5B, left). While labeled neurons were observed in the contralateral arcuate nucleus, a greater number of labeled neurons were found in the side that received the injection. GFP expressing neurons were also identified in the LH, VMH and DMH. At 5 days post-infection, labeled neurons were evident in several extrahypothalamic sites including the BLA, Pir and lateral entorhinal cortex (LEnt) (FIG. 5C, left). Higher magnification of infected neurons in the BLA revealed what may be a synaptic connection between neurons, a possibility that can be confirmed using electron microscopy (FIG. 5C, right). Labeled neurons were also observed in the agranular and granular retrosplenial cortex (FIG. 5D). In this region, the morphology of the GFP expressing cells suggested that both pyramidal neurons (red arrows) and interneurons (white arrow) were infected. Labeled neurons in these brain regions are most likely second order or higher since they appear only at later stages of infection. Fluorescence was restricted to small, discrete numbers of neurons in each of these nuclei and that the infected cells are not contiguous. These data are consistent with previous results indicating that the virus spreads predominantly in a transneuronal fashion rather than laterally (25).

In addition to the sites shown in FIG. 5, labeling was also observed in several other sites. particularly at later times post infection (TABLE 2). At 5 days post-infection, labeling was observed in the perirhinal, entorhinal, and somatosensory 2 cortex. At 7 days, GFP labeled neurons were visible in the somatosensory 1 and retrosplenial cortex as well as in the CA1 region of the hippocampus and brainstem nuclei. While neurons that were first observed at 5d or later are most likely third order or higher, further analyses will be necesssary to determine the precise number of synapses between these sites and the hypothalamus.

Although there were some overlap between the labeled sites observed for the ObRb-Cre and NPY-Cre mice, more brain areas were labeled in the ObRb-Cre mice. This is consistent with the fact that, in the arcuate nucleus, Ob-Rb is co-expressed with NPY, POMC and possibly other neurotransmitters (17). Thus , in the hypothalamus there are several classes of neurons that express ObR, only one of which co-expresses NPY. The GFP+ sites that are seen only in the ObR-Cre mice may have resulted from projections to infected ObR neurons that do not express NPY.

In summary, we have used a conditional Pseudorabies virus to trace the afferent connections of arcuate neurons that express either an abundant transcript, NPY, or a low abundance transcript, Ob-Rb. In many cases our data are consistent with previous studies in which the connections of unspecified neurons were mapped. In other instances, previously unknown connections have been visualized. For example, projections from the BLA, Pir, and BST to the hypothalamus have been reported using other tracing methods, but the connections from the retrosplenial cortex and LEnt to the hypothalamus have not been detected previously (23-25). While our viral tracing experiments revealed a variety of afferent inputs in both NPY- and ObRb-Cre mice it is also possible that additional inputs exist that have escaped detection.

While the kinetics of neuronal labeling can be used to establish a hierarchy of connections, the designation of neurons as 1^{st}, 2^{nd}, 3^{rd} and 4^{th} order etc is not yet confirmed. Further studies including three-dimensional reconstruction of these images should reveal the precise nature of the connections to the hypothalamus from these brain regions.

These data indicate that hypothalamic neurons that express the leptin receptor receive inputs from a number of CNS centers and are likely to integrate signals from these other sites before in turn transmitting impulses to a set of as yet unidentified, efferent sites. Modifications to this viral tracing system using PRV strains that can be transmitted in the anterograde direction, may also make it possible to also trace these efferent connections (26). This method and modifications to it may prove useful in studies of other complex neural circuits.

Materials and Methods

Vector BA2001 Construction

To construct Ba2000, a fragment encoding Tau-GFP (Rodriguez, I. et al. (1999) Cell 97:199) was inserted into XhoI/XbaI sites of pcDNA3. The loxSTOPlox cassette from pBS302 (Gibco-BRL, Gaithersburg, Md.) was generated by PCR and inserted into the KpnI site of the above construct to give the plasmid pLT. The region containing the CMV promoter to the polyA site was then inserted into the BamHu/NotI sites in the PRV genomic subclone pGS202 to give pG2LT. To construct Ba2001, an IRES-TK fragment was ligated into the Xbal site downstream of the Tau-GFP cassette in pG2LT. Cotransfection of BaBlu and transfer vector DNA and blue-white plaque screening was done as previously described (A. Knapp and L. Enquist, J Virol. 71, 2731 (1997)).

Intracerebral Virus Injection

Intracerebral virus injections were performed essentially as described (Card et al. (1999) J. Comp. Neurol. 407:438) with the exception that a 32 gauge needle was used to minimize tissue trauma and fluorescent microspheres (Molecular Probes, Eugene, Oreg.) were coinjected to mark injection site. For striatal injections, injection coordinates were +0.38 mm Bregma, 1.5 mm lateral of saggital suture, and −3.5 mm dorsal-ventral. For cortex injections, coordinates were −1.2 mm Bregma, 3.65 mm lateral and −2.65 dorsal-ventral. Coordinates for arcuate injections were: −1.9 mm Bregma, 0.15 mm lateral, and −5.5 mm dorsal-ventral. Coordinates were determined according to Franklin and Paxinos (K. B. J. Franklin, G. Paxinos (Academic Press, 1997) The Mouse Brain in Stereotaxic Coordinates). Fluorescent blue microspheres (1.0 mm diameter) were obtained from Molecular Probes and added to virus lysate to a concentration of 2%. For virus injections, 100 nl of virus was injected at a rate of 10 nl/minute. Following delivery, needle was removed after a 10 minute period.

Determination of Virus by Plaque Assay

Following injection of virus into the right side of the brain, animals were sacrificed at 6 days post-infection, brains removed and cut saggitally at midline, taking the left side of the brain. Brain tissue was minced, transferred to a 1.5 ml microfuge tube washed 2× with sterile PBS and tissue chunks resuspended in 1.0 ml DMEM with 2% FBS. The tissue was then homogenized and subjected to 3 freeze-thaw cycles. Particulate matter was pelleted and the supernatant used for plaque assays.

Immunofluorescence

Tau-GFP was detected by anti-GFP immunofluorescence and the expression of viral late protein gC was detected using anti-gC antibodies. Animals were sacrificed by pentobarbital overdose and perfused with 4% paraformaldehyde in phosphate buffered saline (PBS). Brains were post-fixed, equilibrated in PBS+30% sucrose and sectioned serially on a cryostat. For immunofluorescence, sections were blocked in PBS with 2% goat serum, 3% BSA, 0.1% triton X-100 and incubated in the respective primary antibody according to the supplier's recommendations. Polyclonal anti-GFP antibodies were obtained from Molecular Probes. Anti-NPY antibodies were obtained from Peninsula Labs (San Carlos, Calif.). Monoclonal anti-gC antibodies were purchased from Chemicon (Temecula, Calif.). Sections were then washed and incubated in FITC or TRITC secondary antibody. Primary antibody dilutions used were: anti-gC, 1:1000; anti-NPY, 1:10,000; anti-GFP, 1:350. Sections were examined using a Zeiss axioplan microscope. Images were collected using a Princeton Graphics digital camera and processed using IPLab Powermicrotome deconvolution software from Scanolytics.

Generation of BAC Transgenics Expressing Cre Recombinase

BAC clones containing the NPY gene were identified by hybridization to a mouse BAC library filter array using a mouse NPY cDNA fragment. A pSV1 shuttle vector subclone containing the NPY exons 1 and 2 was subsequently generated to give pSVl-NPY. Using PCR, the first ATG was mutated to ATT and a PacI site was introduced directly 3′ to the mutated ATG. PCR was used to generate an NLS-Cre-SV40 polyA which was then inserted into the PacI site of pSV1-NPY. Recombination into the BAC clone was performed as described (Yang et al. (1997) Nature Biotechnol. 15: 859).

Discussion

A fuller understanding of the neural mechanisms underlying complex behaviors will in part require a clear representation of the proximal and distal connections of the CNS centers that control that behavior. Our results establish that an alpha-herpes virus conditional both for replication and marker protein expression can be used for transynaptic tracing of the afferent synaptic connections of specific neurons. The strain of virus described here, Ba2001, is dependent on the expression of Cre recombinase for expression of TK (required for viral replication in non-mitotic cells) and for expression of the GFP marker protein replication expression of the Tau-GFP marker protein.

In these experiments, Ba2001 was used to trace the afferent connections of arcuate neurons that express either an abundant transcript NPY or a low abundance transcript, the long form of the leptin receptor. In many cases these data are consistent with previous studies in which tracing of unidentified neurons was performed. In other instances, previously unknown connections have been visualized. Thus while projections from the BLA, Pir, and PBN to the hypothalamus have been reported using other tracing methods, connections from the retrosplenial cortex and LEnt to the hypothalamus had not (25). It is as yet unclear which of the neurons are 1st, 2nd, 3rd and 4th order etc. Further studies including three-dimensional reconstruction of these images should reveal the precise nature of the connections to the hypothalamus from these brain regions.

The NPY and ObRb expressing neurons are known to play a role in regulating food intake and body weight. These data indicate that, in addition to leptin, these cells receive inputs from a number of other brains regions including the cortex. The nature of the information that is conveyed by these inputs is as yet unknown. The piriform, entorhinal and perirhinal cortices are known to receive projections from the olfactory cortex so it is possible that olfactory stimuli influence leptin signaling. These same regions in turn receive (and send) projections from the hippocampus suggesting that evoked memories, perhaps related to odor and food identification, may also have an impact on feeding behavior via a series of connections through the amygdala to neurons expressing the leptin receptor in the hypothalamus. Projections from the amygdala may also represent a pathway by which emotion could influence food intake.

In aggregate these data indicate that hypothalamic neurons that express the leptin receptor receive inputs from a number of CNS centers and may serve an integratory function. Thus it is likely that these ObRb expressing neurons sense plasma leptin level and signals from these other inputs before transmitting impulses via a series of, as yet unidentified, efferent connections. Modifications to this viral tracing system may make it possible to also trace these efferent connections. While the Bartha strain spreads predominantly by retrograde transport, less virulent derivatives of the Becker strain can spread both retrograde and anterograde. Thus the creation of analogous PRV strains on the Becker background cquld make it possible to also trace the efferent connections of these (and other) neurons.

We expect that Ba2001 will prove useful in studies of other neural circuits. The use of the PRV vector for neural tracing has several useful features relative to other tracing methods including the recently reported use of WGA in transgenic mice (27). Some of the notable features of the method reported here are as follows: (i) the tracing studies can be limited to discrete regions by injecting the virus sterotaxically. The use of transgenic mice expressing Wheat germ agglutinin leads to expression of the marker at all sites where the gene is expressed. This could lead to complex patterns of expression for widely expressed genes such as NPY; (ii) the system allows tracing of multiple order neurons using even low abundance transcripts, such as ObR, to drive Cre synthesis. Successful tracing using wheat germ agglutinin is at present limited to strong promoters and its ability to trace 2nd (or more) order neurons is limited; (iii) the use of the Bartha strain allows retrograde tracing while what germ agglutinin is useful for anterograde tracing. While these methods are complementary, it should also be noted that, as mentioned above, Becker strains can be used for anterograde tracing (26); (iv) At present only a limited number of agglutinin molecules are available for tracing. In contrast a potentially limitless number of markers could be inserted into the viral genome; (v) Analysis of sections at different time points following infection should make it possible to distinguish first, second, etc., order neurons can be distinguished. Such information could then be used to construct a hierarchical map of the synaptic connections.

The methods described allow the tracing of the distant connections of specific classes of neurons. It is as yet unclear exactly how many synapses can be traced although it appears that in some cases 4th order neurons can be identified (data not shown). The ability to trace beyond this point may be limited by the life span of the animal. Death from PRV is often the result of an intense immune reaction to the infection. While animals seldom lived past seven days in these studies, construction of less virulent strains and pharmacological interventions such as immunosuppression could further extend the length of time that an experiment could be continued and allow the detection of even more distant connections. Mortality after PRV infection is often the result of a robust immune response to the virus.

In summary, we report a novel method has been used to trace the inputs to specific neurons in the hypothalamus. These data revealed a number of heretofore unappreciated connections between higher centers and the hypothalamus and further suggest that neurons in this region serve a key integratory function in regulating food intake and body weight. This method may prove useful in studies of other complex neural circuits.

### EXAMPLE 2

The use of neurotropic viruses, including PRV, to trace and characterize neuronal pathways can be extended and varied in a number of other ways. Examples of additional conditional expression schemes and PRV constructs to be utilized, including bicistronic and monocistronic constructs are provided in FIGS. 7-10.

Neurotropic viruses may be utilized that substitute different GFP variants, which are distinguishable with the appropriate fluorescence filters (28). In this way, the spatial organization of neurons in nuclei that receive inputs from a number of distinct sites could be mapped both in that nucleus and at the level of the inputs to that nucleus. The extent of overlap of pathways would be identifiable by double labeling.

Alternative site-specific recombinases (e.g., FLP, A integrase, R recombinase, Kw recombinase) may be utilized to construct a distinct series of recombinase-dependent viruses. By utilizing a virus expressing a GFP protein that was distinguishable from that of Ba2001 along with Ba2001, it would be possible to trace simultaneously different pathways in animals that express Cre in some neurons and Flp in others.

While the Bartha strain utilized in Example 1 spreads predominantly by retrograde transport, less virulent derivatives of the Becker strain can spread both retrograde and anterograde. Thus the creation of analogous PRV strains on the Becker background could make it possible to also trace the efferent connections of these (and other) neurons. By labeling a Becker construct with a distinct GFP or other marker (e.g. epitope tag marker) and utilizing it in concert with a retrograde virus construct (e.g. Ba2001), the double labeled pathway would mark retrograde tracing, while the singly Becker labeled tracing would mark anterograde.

PRV viruses, or other herpesviruses, can be constructed that carry mutations in viral genes that regulate expression of host RNAs. For example, UL41 is the PRV homologue of the herpes simplex virus virion host shut off gene (vhs) whose function is thought to be to degrade mRNA during infection (29,30). In the absence of vhs function, host genes are continually expressed on infection of a UL41 or other vhs equivalent mutant. Infection of Cre expressing animals with a UL41 mutant would therefore make it possible to determine the neuron's transcriptional profile using DNA microarrays, particularly facilitated by the expression of a marker peptide, for instance a fluorescent label or epitope tag, which identifies the PRV infected cell(s). It would also be possible, by virtue of the fluorescent label or epitope tag marker, to sort (e.e., FACs sort) and thereby isolate the virus-infected cells for transcriptional profile analysis.

Design and utilization of a conditionally lethal virus provides the ability to specifically ablate or disrupt cells in a neural pathway. These viruses conditionally express a toxin, apoptotic protein or other agent that terminates the virus life cycle and/or causes cell death in the virus infected cell. This system results in the destruction of a portion of the neural circuit. Analysis of the resulting phenotype of an animal infected with such a conditionally lethal virus would provide insight regarding the function of the affected neurons. These animals may provide unique models for conditions, particularly neural conditions, wherein a specific neural circuit or response pathway is altered or obliterated. In a conditional lethal virus construct conditionally expressing an attenuated form of diphtheria toxin, for instance in a Cre-dependent manner, activation of diphtheria toxin expression halts all protein synthesis in the cell resulting in the cell's death. Alternatively, an apoptotic protein may be conditionally expressed, leading to specific neural cell death on virus infection and activation. CAG trinucleotide expansion leads to neural cell death in certain diseases. Proteins with expanded CAG repeats may be conditionally expressed to generate cell death via conditionally lethal virus constructs. Spinocerebellar ataxia type 3 (SCA3) is an autosomal dominantly inherited neurologic disorder characterized by neuronal loss and gliosis. SCA3 is caused by a CAG trinucleotide/polyglutamine repeat expansion in the SCA gene. Ataxin-3 protein is encoded by the SCA3 gene. High level expression of expanded full-length ataxin-3 in neural cell lines results in spontaneous non-apoptotic cell death (Evert, B. D. et al (1999) Hum Molec Genet 8(7): 1169-1176). Huntington's disease (HD) is similarly caused by unstable expansion of CAG trinucleotide repeats. Transiently transfected cells with different sized HD with expanded CAG repeats demonstrate increased cell death (Martindale, D. et al (1998) Nature Genet 18:150-154; Saudou, F. et al (1998) Cell 95:55-66; Lunkes A. and Mandel, J. L. (1998) Hum Molec Genet 7:1355-1361).

Examples of vectors for use as a conditionally lethal system are depicted in FIGS. 11 and 12.

### EXAMPLE 3

While conditional expression of GFP and TK can be achieved using the Ba2001 strategy described above, the Tet-Off system has been utilized in an effort to maximize marker protein expression (GFP, etc.). As depicted in FIG. 13, a LoxP stem loop structure generated after recombination in the earlier strategy can impede translation initiation. In this system, the virus expresses the transactivator Tet-Off only after modification by a recombinase (Cre, FLP, λ recombinase, R recombinase, Kw recombinase, etc. produced by specific cells in transgenic or recombinant animals as described above). Tet-Off, in the absence of its allosteric inhibitor, binds to the Bi promoter which contains Tet-Off binding sites flanked by TAT-boxes and CMV immediate early proximal promoter regions. This results in very high levels of GFP marker expression and TK, significantly higher than that seen in Ba2001. Exemplary constructs for this Tet-Off system and results are depicted in FIGS. 14 and 15.

An additional and perhaps equally important feature of this system is that it facilitates the screening for recombinant viruses during the final stages of virus construction. Following transfection of the DNA construct and PRV DNA, the resulting lysate can be plaqued onto a stably transfected cell line that constitutively expresses Tet-Off (for example the PK-15 cell line). Since GFP expression (and fluorescence) is dependent on the presence of Tet-Off protein, viruses carrying the insert of interest (and their plaques) are rapidly and readily identified by fluorescence microscopy. The trans-complementation by the cell's own Tet-Off protein is temporary, with the virus' genome remaining unaltered. This system and a representative construct are depicted in FIG. 16.

### EXAMPLE 4

In order to determine whether circuits innervating peripheral targets such as adipocyte tissue (“fat pads”) or liver involve neurons that express OB receptor (ObRb) or other gene of interest, a recombinant Bartha virus that is conditional for GFP expression only has been used (BaLT recombinant construct depicted in FIG. 18. Since hypothalamic neurons are not known to directly innervate these peripheral targets (FIG. 17), a replication-competent virus must be used that can cross several synapses before reaching a Cre-expressing neuron. Following injection into epidiymal fat pads of ObRb-Cre mice, GFP expressing neurons are found first in the hypothalamus and later in the amygdal and cortex (FIG. 19). GFP expression is dependent on the presence of Cre since it is absent in wild type animals (not shown). No GFP expressing virus is found in the spinal cords of mice at any stage of infection, indicating that the virus is being recombined by Cre only in the CNS (FIG. 20). Similar results have been found in ObRb Cre mice after receiving liver injection of virus (not shown).

### REFERENCES

1. J. M. Friedman, J. L. Halaas, Nature 395, 763-770 (1998).

2. J. L. Halaas et al., Proc. Natl. Acad. Sci. USA 94, 8878-8883 (1997).

3. J. K. Elmquist, R. S. Ahima, C. F. Elias, J. S. Flier, C. B. Saper, Proc Natl Acad Sci USA 95, 741-6 (1998).

4. A. D. Loewy, Neuroscience & Biobehavioral Reviews 22, 679-84 (1998); L. W. Enquist, P. J. Husak, B. W. Banfield, G. A. Smith, Adv Virus Res 51, 237-347 (1998).

5. J. P. Card, L. W. Enquist, R. Y. Moore, Journal of Comparative Neurology 407, 438-52 (1999).

6. A. Standish, L. W. Enquist, R. R. Miselis, J. S. Schwaber, Journal of Neurovirology 1, 359-68 (1995).

7. M. Lakso et al., Proc Natl Acad Sci U S A 89, 6232-6 (1992).

8. T. Maniatis, E. F. Fritsch, J. Sambrook, Molecular Cloning—A Laboratory Manual (Cold Spring Harbor Laboratory, 1982).

9. Y. Gordon et al., Archives of Virology 76, 39-49 (1983).

10. C. Logvinoff, A. L. Epstein, Virology 267, 102-10 (2000).

11. G. A. Gentry, J. F. Aswell, Virology 65, 294-6 (1975).

12. J. Marth, personal communication.

13. A. K. Robbins, R. J. Watson, M. E. Whealy, W. W. Hays, L. W. Enquist, Journal of Virology 58, 339-47 (1986).

14. L. W. Enquist, J. Dubin, M. E. Whealy, J. P. Card, Journal of Virology 68, 5275-9 (1994).

15. A. M. Strack, A. D. Loewy, Journal of Neuroscience 10, 2139-47 (1990).

16. Y. Hosoya, Y. Sugiura, N. Okado, A. D. Loewy, K. Kohno, Experimental Brain Research 85, 10-20 (1991).

17. J. C. Erickson, G. Hollopter, R. D. Palmiter, Science 274, 1704 (1996); H. Higuchi, H. Y. Yang, S. L. Sabol, J Biol Chem 263, 6288-95 (1988).

18. C. Elias et al., Neuron 23, 775 (1999).

19. D. R. Gehlert, B. M. Chronwall, M. P. Schafer, T. L. O'Donohue, Synapse 1, 25 (1987).

20. L. Zaborsky, Afferent connections of the medial basal hypothalamus. W. Hild, Ed. (Springer-Verlag, New York, 1982), vol. 69.

21. H. Fei et al., Prod. Natl. Acad. Sci., USA. 94, 7001-7005 (1997).

22. J. P. Card et al., J. of Neuroscience 10, 1974 (1990).

23. F. C. Barone, M. J. Wayner, S. L. Scharonn, R. Guevara-Aguilar, H. U. Aguilar—Baturoni, Brain Res. Bull. 7, 75 (11981).

24. H. Bester, J. M. Besson, J. F. Bernard, J. Comp.Neurol. 383, 245 (1997).

25. G. D. Petrovich, P. Y. Risold, L. W. Swanson, J. Comp Neurol. 374, 387 (1996).

26. M. Yang, J. P. Card, R. S. Tirabassi, R. R. Miselis, L. W. Enquist, J. of Virol. 73, 4350 (1999).

27. Y. Yoshihara et al., Neuron 22, 33-41 (1999); L. F. Horowitz, J. P. Montmayeur, Y. Echelard, L. B. Buck, Proc Natl Acad Sci U S A 96, 3194-9 (1999).

28. R. Heim, R. Y. Tsien, Curr Biol 6, 178-82 (1996).

29. H. Berthomme, B. Jacquemont, A. Epstein, Virology 193, 1028-32 (1993); G. S. Read, B. M. Karr, K. Knight, J Virol 67, 7149-60 (1993).

30. G. S. Read, B. M. Karr and K. Knight, J Virology 67, 7149-7160 (1993).

This invention may be embodied in other forms or carried out in other ways without departing from the spirit or essential characteristics thereof. The present disclosure is therefore to be considered as in all aspects illustrate and not restrictive, the scope of the invention being indicated by the appended claims, and all changes which come within the meaning and range of equivalency are intended to be embraced therein.

Various references are cited throughout this Specification, each of which is incorporated herein by reference in its entirety.

What is claimed is:

1. A method for characterizing a neuronal pathway in an animal which utilizes a neurotropic virus capable of spreading between synaptically connected neurons, wherein said virus is conditional for marker peptide expression, whereby said marker peptide is expressed upon a specific recombination event and/or in the presence of an inductive agent.

2. A method for characterizing a neuronal pathway in an animal comprising:
a) introducing a neurotropic alphaherpesvirus capable of spreading between synaptically connected neurons which is conditional for marker peptide expression, whereby said marker peptide is expressed upon a specific recombination event;
b) introducing or activating a recombinase such that said recombination event is induced and marker peptide expression occurs; and
c) monitoring marker peptide expression.

3. The method of claim 2 wherein said neurotropic alphaherpesvirus is also conditional for replication, whereby replication is permissive upon a specific recombination event, further comprising:
a) introducing or activating a recombinase such that said recombination event is induced and replication of said neurotropic virus occurs.

4. The method of claim 3, wherein both marker protein expression and replication are conditional upon the same specific recombination event.

5. A method for characterizing a neuronal pathway in an animal comprising:
a) introducing a neurotropic alphaherpesvirus capable of spreading between synaptically connected neurons which is conditional for marker peptide expression, whereby said marker peptide is expressed in the presence of an inductive agent;
b) introducing said inductive agent such that marker peptide expression occurs; and
b) monitoring marker peptide expression.

6. The method of claim 5, wherein said neurotropic alphaherpesvirus is also conditional for replication, whereby replication is permissive upon a specific recombination event, further comprising:
a) introducing or activating a recombinase such that said recombination event is induced and replication of said neurotropic virus occurs.

7. The method of any of claims 2, 3 or 6 wherein said recombinase is from prokaryotic or eukaryotic cells.

8. The method of claim 7 wherein said recombinase is selected from the group of Cre, FLP, λ integrase, R recombinase or Kw recombinase.

9. The method of any of claims 2, 3 or 6 wherein the expression of the recombinase is under the control of a heterologous expression control sequence.

10. The method of claim 9 wherein the heterologous expression control sequence is neural cell specific.

11. The method of claim 10 wherein the heterologous expression control sequence is that of a neuropeptide or neuroactive peptide.

12. The method of claim 11 wherein the heterologous expression control sequence is selected from the group of NPY and Ob-R.

13. The method of any of claims 2, 3, 4, 5, or 6 wherein the neurotropic alphaherpesvirus is introduced by a means selected from the group of intranasal infection, injection into the brain of said animal, injection or infusion into the spinal cord of said animal, or injection into a peripheral organ of said animal.

14. A recombinant neurotropic virus, capable of tracing neural connections across multiple synapses and capable of conditionally expressing a marker peptide upon a specific recombination event and/or in the presence of an inductive agent.

15. The virus of claim 14 which is a neurotropic alphaherpesvirus.

16. The virus of claim 15 which is a pseudorabies virus.

17. The virus of claim 16 which strain of pseudorabies virus is selected from the group of Bartha, Becker or Kaplan.

18. The virus of claim 17 which is further selected from the group of Ba2000 or Ba2001.

19. A transgenic animal harboring the virus of claim 14.

20. A transgenic animal harboring the virus of claim 14, wherein said virus is also conditional for replication upon a specific recombination event and/or in the presence of an inductive agent.
